(19) United States
(12) Patent Application Publication
(10) Pub. No.: US 2023/0233737 A1
(43) Pub. Date: Jul. 27, 2023

(54) IMPLANTABLE PROSTHETIC DEVICES HAVING OUTER SHELLS WITH INTEGRATED SCAFFOLDING FOR IMPROVING FORM STABILITY, REDUCING WRINKLING AND REDUCING THE WEIGHT OF THE IMPLANTABLE PROSTHETIC DEVICES

(71) Applicant: Mentor Worldwide LLC, Irvine, CA (US)

(72) Inventors: Joseph Henry Contiliano, Stewartsvile, NJ (US); Michael Hoffman, Hillsborough, NJ (US); Lucas Heldman, Wayne, NJ (US); Vikram Garadi, Irving, TX (US); Sriram Natarajan, Hillsborough, NJ (US)

(21) Appl. No.: 17/965,961

(22) Filed: Oct. 14, 2022

Related U.S. Application Data
(60) Provisional application No. 63/302,692, filed on Jan. 25, 2022.

(51) Int. Cl. A61L 27/18; A61L 27/52

(52) U.S. Cl. CPC A61L 27/18; A61L 27/52; A61L 2430/04

(57) ABSTRACT

An implantable prosthetic device, such as a breast implant, includes a shell made of a biocompatible elastomeric material. The shell has a front portion and a base that surround an interior volume of the shell. A scaffold is disposed within the interior volume of the shell. The scaffold has an inner surface facing the base and an outer surface facing the front portion of the shell. A biocompatible filler material, such as a silicone gel, is disposed within the interior volume of the shell. The scaffold has a shape that mirrors the shape of the front portion of the shell. The scaffold reinforces the shell to provide form stability for maintaining the shape of the shell and minimizing folding, dimpling and/or wrinkling of the shell. The scaffold has one or more openings formed therein for allowing the biocompatible filler material to fill the one or more openings. A second scaffold may be nested within the first scaffold. The second scaffold has a smaller outer dimension than an inner dimension of the first scaffold.

### CROSS-REFERENCE TO RELATED PATENT APPLICATIONS

The present patent application claims benefit of U.S. Provisional Application Serial No. 63/302,692, filed on Jan. 25, 2022, the disclosure of which is hereby incorporated by reference herein.

### BACKGROUND OF THE INVENTION

### Field of the Invention

The present patent application is generally related to medical devices, and is more specifically related to implantable prosthetic devices such as gel breast implants.

### Description of the Related Art

Implantable prosthetic devices, such as breast implants, are commonly used to replace or augment body tissue. In the case of the female breast, it may become necessary to remove some or all of the mammary gland and surrounding tissue in order to treat breast cancer. This surgery typically leaves a void that may be filled with an implantable prosthetic device that supports surrounding tissue and provides a normal body appearance, eliminating much of the shock and depression that often follows breast cancer surgeries. Implantable prosthetic devices are also used for breast augmentation procedures.

Implantable prosthetic devices usually include a shell made of silicone or a biocompatible polymer. Shells are typically manufactured by dipping an appropriately sized and shaped mandrel into silicone. The mandrel may be solid or hollow. In other methodologies, a silicone solution may be sprayed onto the mandrel and allowed to cure. Hollow molds may also be used for forming the shells of implantable prosthetic devices.

When a mandrel is used for making an implant, the process results in the formation of a shell having a mandrel opening, e.g., a circular hole, in one of its faces. After the shell has been formed, it must be removed from the mandrel. The mandrel opening is subsequently covered with a patch that seals the hole to form a fluid impervious implant shell. The completed shell can remain unfilled, be pre-filled, or intraoperatively filled through a small fill port or valve with a solution such as gel, saline, foam, or combinations of these materials.

In some instances, silicone breast implants are not completely filled with solution. This situation may result in the formation of a dimple or concavity at the apex of the implant, which is commonly referred to as the ashtray effect. The ashtray effect is frequently most evident when the implant is positioned atop a flat surface.

FIG. 40 shows a conventional breast implant 50 having an apex 52 at an upper end thereof, a base 54 at a lower end thereof, a radius 56 that extends around the circumference of the implant, and a dome 58 having a convexly curved surface that extends between the apex 52 and the radius 56. Depending upon the amount of gel within the shell, the astray effect may result (i.e., the presence of dimples, depressions, or concavities at the apex). Many efforts have been directed to eliminating the ashtray effect including upside down curing and adding extra gel. Both approaches may increase the cost of an implant or involve expensive tooling. Adding extra gel may also add additional weight to the implant, which is not desirable. Moreover, in many instances, upside down curing has not been deemed to efficiently remove the ashtray effect.

Breast implants are generally designed to be relatively soft and pliable, which make breast implants susceptible to rippling or wrinkling. FIG. 41 shows a breast implant 70 having ripples 72. Avoiding or minimizing the occurrence of ripples has become an issue of enhanced importance with the increase of pre-pec procedures whereby implants are placed above the pectoralis muscle and closer to the skin. In any event, wrinkles and rippling are not desirable and technologies to reduce or eliminate their occurrence are sought without increasing the amount of gel, increasing shell tension, and/or increasing the outer diameter of the implant.

Another undesirable occurrence that arises with mammary implants is the formation of wrinkles along one or more edges of the implant, which is commonly referred to as scalloping. Referring to FIG. 42, a conventional implant 80 has an upper pole 82 and a lower pole 84. Several creases 86 are shown on the upper pole 82 of the anterior face. The creases 86 (i.e., scalloping) radiate inwardly from the perimeter 88 of the prosthetic device. The creases formed in the anterior face can be discerned through the skin of the patient and are not aesthetically desirable.

In many instances it is desirable to create implant devices that maintain or increase the projection of implants without requiring an increase in the amount of gel or the gel/shell ratio. Increasing gel undesirably adds additional weight, and increases the tension on the shell.

Referring to FIG. 43, in order to avoid the ashtray effect, rippling, wrinkling, and/or scalloping, and in order to improve the projection of the apex of the shell, some breast implant manufacturers provide breast implants 90 that are more fully filled with gel or saline solution. Many conventional implants contain about 400 cc of gel or saline. In one design, an additional 65 cc of gel or saline is introduced into the shell. The additional gel or saline added to the shell of the implant 90 improves the projection of the apex 92 of the implant. Unfortunately, increasing the volume of gel or saline within the implant 90 adds additional weight to the implant, and increases the tension on the shell. The additional weight may lead to problems such as back pain for patients.

The fatigue strength of a shell and/or implant is an important characteristic for providing for a long product life. One way to increase fatigue strength is to increase shell thickness, however, this may adversely affect the natural feel of the implant as thinner shells typically feel more natural. There is a continuing need for implant shells having improved fatigue strength while maintaining normal shell thickness and a more natural feel.

The form stability (i.e., the ability of an implant to maintain its shape) is an important consideration. Current means to afford improved form stability are directed toward increasing the cohesiveness of the gel that fills the implant. Increased gel cohesiveness changes the feel of the implant to less soft and less natural.

Conventional prosthetic implants typically include a silicone shell that is filled with a silicone gel. The shell is designed to be soft and not structurally rigid. The silicone gel that is used to fill the shell tends to bond to the inner surface of the shell, whereupon the gel can pull on the outer wall of the shell, which creates undesirable folds, wrinkles and/or dimples in the shell.

At present, there are very few technologies available improving the form stability of breast implants. Two well-known methods used to improve form stability include using more cohesive gels and increasing the volume of gel that is used to fill the shell. Unfortunately, both of these methods tend to increase the firmness of the implant and/or the weight of the implant.

There have been some successful efforts directed to improving the form stability of breast implants. For example, U.S. Pat. No. 10,898,313, assigned to Mentor Worldwide LLC, the disclosure of which is hereby incorporated by reference herein, discloses an implantable prosthetic device including a shell having an apex, a base, a radius located between the apex and the base, and a dome extending between the apex and the radius. The shell has an outer surface and an inner surface that surrounds an interior volume of the shell. One or more ribs are integrally formed with the inner surface of the shell and project inwardly from the inner surface of the shell into the interior volume of the shell. The interior volume of the shell is filled with a biocompatible filler material, such as silicone gel. The one or more ribs function as shell stiffeners to enhance the structural integrity and stability of the shell, to enhance the projection of the apex of the shell, and to minimize the occurrence of the ashtray effect, rippling, wrinkling, and/or scalloping.

In spite of the above advances, there remains a need for mammary implants that minimize the occurrence of the ashtray effect, rippling, wrinkling, and scalloping, while providing improved projection at the apex of the shell. In addition, there remains a need for systems, devices and methods that minimize the weight of implantable prosthetic devices. There also remains a need for implants having enhanced structural integrity, and that maintain a soft feel to the touch.

### SUMMARY OF THE INVENTION

In one embodiment, an implantable prosthetic device, such as a breast implant, preferably includes a shell made of a biocompatible elastomeric material (e.g., silicone), the shell having a front portion and a base that cooperatively surround an interior volume of the shell.

In one embodiment, the biocompatible elastomeric material used to make the shell may be silicone or a polymer.

In one embodiment, the implantable prosthetic device desirably includes a scaffold that is disposed within the interior volume of the shell. The scaffold may be made of a polymeric material such as silicone. In one embodiment, the scaffold has an inner surface that faces toward the base of the shell and an outer surface that faces toward the front portion of the shell.

In one embodiment, a biocompatible filler material (e.g., silicone gel) may be disposed within the interior volume of the shell that surrounds the inner and outer surfaces of the scaffold.

The scaffolds disclosed in the present patent application preferably improve form stability or the ability of an implant to maintain its shape. The scaffolds disclosed herein preferably increase strength and rigidity without increasing the shell wall thickness, thus maintaining softness while improving form stability.

In one embodiment, with the scaffold disposed inside the outer shell, the outer shell is filled with the biocompatible filler material (e.g., a gel; a silicone gel) that contacts and/or adheres to one or more surfaces of the scaffold.

In one embodiment, the scaffold may be placed within the biocompatible filler material of the implantable prosthetic device prior to curing the biocompatible prosthetic material.

In one embodiment, the scaffold is designed to replace a small portion (by weight) of the biocompatible filler material (e.g., silicone gel) that fills the outer shell of the implantable prosthetic device.

In one embodiment, the scaffold may be positioned symmetrically within the center of the implant.

In one embodiment, the scaffold may be positioned (e.g., centered) over a base or rear wall of the outer shell.

In one embodiment, the scaffold has sufficient mechanical integrity and bonds to the biocompatible filler material in such a way that it “scaffolds” the biocompatible filler material (i.e., reduces the ability of the biocompatible filler material to flow freely within the shell). By affecting the ability of the biocompatible filler material to flow freely within the shell, the scaffold helps to maintain the shape of the filled implant device, thereby enhancing the form stability of the implant.

In one embodiment, the scaffold provides form stability for a gel-filled implant by maintaining the shape of the gel-filled implant, such as after the implant has been rotated 90 degrees into a vertical orientation (i.e., the position assumed when a woman with breast implants stands up.) Thus, the shell of the gel-filled implant will maintain its original shape when it is rotated from a horizontal configuration (i.e., sitting on flat surface) to the vertical orientation.

In one embodiment, the scaffold may be made of silicone. In other embodiments, the scaffold may be made of other polymeric materials, particularly those that will bond with the biocompatible filler materials used to fill the shell of the implant.

Incorporating the internal scaffolding disclosed herein into an outer shell of an implantable prosthetic device enables the implant device to be placed into a vertical orientation while maintaining the form stability of the implant, with demonstrably reduced observable wrinkles, creases, or folds forming on the surface of the outer shell.

The internal scaffolding also provides form stability without having to increase the cohesiveness of the gel or the volume of gel that is introduced into the shell, thus minimizing the firmness of the device and minimizing the overall weight of the device. Heavier implant devices may increase the potential for back issues (e.g., back pain) due to the extra weight of the implant. Using lighter implants helps to avoid the problems associated with heavier devices.

In one embodiment, the scaffold has a concave inner surface that faces toward the base of the shell. Many different scaffold geometries that have a concave inner surface may be utilized. In one embodiment, the biocompatible filler material (e.g., a cohesive filler material; a silicone gel) that fills the concave geometry of the scaffold preferably acts like a honeycomb to limit the flow of the biocompatible filler material within the shell.

In one embodiment, the biocompatible filler material disposed within the interior volume of the shell is adhered to at least a portion of the inner surface or the outer surface of the scaffold.

In one embodiment, the inner surface of the scaffold may be concave, and the outer surface of the scaffold may be convexly curved.

In one embodiment, the convexly curved outer surface of the scaffold preferably mirrors the shape of the front portion of the shell.

In one embodiment, the scaffold may have one or more openings (e.g., round holes; slits; squares; rectangles) formed therein that extend from the inner surface of the scaffold to the outer surface of the scaffold. Those skilled in the art may recognize that the one or more openings can be of any known geometry (e.g., round; square; rectangular; elongated slit; triangle).

In one embodiment, the scaffold is attached to the base of the shell.

In one embodiment, the scaffold may have a general shape that is akin to the appearance of a hemisphere having an open base at a lower end thereof. In one embodiment, the scaffold may have a concave shape. In one embodiment, the scaffold may have a concave inner surface and a convexly curved outer surface.

In one embodiment, the scaffold has a lower end including a circular, free edge that surrounds the open base of the scaffold.

In one embodiment, the scaffold may have an inwardly projecting rim that is located at the base (i.e., lower end) of the scaffold and that extends around an opening formed in the base of the scaffold. The scaffold may include a wall having a curvature that extends between the radius or side of the scaffold and the rim that projects inwardly at the base of the scaffold.

In one embodiment, when the scaffold is positioned inside the shell, the circular, free edge of the scaffold is juxtaposed with the base of the shell.

In one embodiment, the circular, free edge of the scaffold may be attached to the base of the shell.

In one embodiment, the circular, free edge of the scaffold is not attached to the base of the shell.

In one embodiment, the front portion of the shell preferably includes an apex and a dome that extends between the apex and the base of the shell.

In one embodiment, the scaffold that is disposed inside the shell preferably has a shape that mirrors the shape of the dome of the shell.

In one embodiment, an implantable prosthetic device may include a second scaffold that is nested within the first scaffold.

In one embodiment, the second scaffold is located between the inner surface of the first scaffold and the base of the shell.

In one embodiment the first scaffold has an inner dimension that is larger than the outer dimension of the second scaffold that is nested inside the first scaffold.

In one embodiment, the first scaffold has a greater height than the second scaffold.

In one embodiment, the base of the first scaffold has an outer diameter that is greater than the outer diameter of the open base of the second scaffold.

In one embodiment, the second scaffold has one or more openings formed therein that extend from an inner surface to an outer surface of the second scaffold.

In one embodiment, the biocompatible filler material that is used to fill the shell is desirably in contact with at least one of the inner and outer surfaces of the second scaffold.

In one embodiment, the front portion of the shell has a shell wall thickness, the first scaffold has a first scaffold wall thickness, and the second scaffold has a second scaffold wall thickness.

In one embodiment, the second scaffold wall thickness is greater than the first scaffold wall thickness. In one embodiment, the wall thicknesses of the respective first and second scaffolds may be the same or similar.

In one embodiment, the first scaffold wall thickness is greater than the shell wall thickness. In one embodiment, the first scaffold wall thickness is the same as the shell wall thickness.

In one embodiment, the second scaffold may have a geometric shape that is different than the geometric shape of the first scaffold or the geometric shape of the front portion of the shell.

In one embodiment, the first scaffold may have a geometric shape that is different than the geometric shape of the second scaffold or the geometric shape of the front portion of the shell.

In one embodiment, the shell has a shell wall thickness, and the scaffold has a scaffold wall thickness that is greater than the shell wall thickness.

In one embodiment, the scaffold has an apex, a radius, and a dome that extends between the apex of the scaffold and the radius of the scaffold.

In one embodiment, the scaffold has a wall that is thicker at the apex of the scaffold and thinner at the radius of the scaffold.

In one embodiment, an implantable prosthetic device preferably includes a silicone shell having a front wall portion and a base that surround an interior volume of the shell.

In one embodiment, a silicone scaffold is disposed within the interior volume of the silicone shell.

In one embodiment, the silicone scaffold desirably has a concave inner surface that faces toward the base of the silicone shell and a convexly curved outer surface that faces toward the front wall portion of the silicone shell.

In one embodiment, a silicone gel is disposed within the interior volume of the shell. The silicone gel preferably fills the silicone shell and surrounds the silicone scaffold.

In one embodiment, the silicone scaffold has one or more openings formed therein that extend from the concave inner surface of the silicone scaffold to the convexly curved outer surface of the silicone scaffold.

In one embodiment, the silicone scaffold has the shape of a hemisphere having an open base and a lower, free edge that surrounds the open base.

In one embodiment, the lower, free edge of the silicone scaffold may have the shape of a circle.

In one embodiment, the lower, free edge of the silicone scaffold is juxtaposed with the base of the silicone shell.

In one embodiment, the lower, free edge of the silicone scaffold is attached to the base of the silicone shell.

In one embodiment, the implantable prosthetic device preferably includes a second silicone scaffold that is nested within the first silicone scaffold and that is located between the concave inner surface of the first silicone scaffold and the base of the silicone shell.

In one embodiment, the front wall of the silicone shell desirably includes an apex, and a dome that extends between the apex and the base of the silicone shell.

In one embodiment, the silicone scaffold has a geometric shape that mirrors the geometric shape of the dome of the silicone shell.

In one embodiment, the front wall portion of the silicone shell has a first wall thickness, and the silicone scaffold has a second wall thickness that is greater than the first wall thickness.

In one embodiment, the shells may be made by dipping or spraying a mandrel with a biocompatible, curable material such as silicone, polymers, polyurethane, silicone-polyurethane co-polymers, elastomers or combinations thereof. After application of the biocompatible, curable material to the mandrel, the curable material is allowed to cure, and the cured shell is removed from the mandrel.

In one embodiment, the scaffold may be formed from the same material as the shell.

In one embodiment, the scaffold may be formed from a different material than that of the shell (e.g., a different silicone or the same silicone further cross-linked).

In one embodiment, the scaffold may be a composite of a second material that is embedded within the scaffold (e.g., a monofilament or multifilament structure that is either polymeric (e.g., suture material) or metallic (e.g., a thin wire).

In one embodiment, the second material may be embedded during a layering or molding process used to form the scaffold.

In one embodiment, the shell has an outer surface that is smooth and an inner surface that surrounds an interior volume of the shell.

In one embodiment, a biocompatible filler material (e.g., gel) is disposed within the interior volume of the shell. In one embodiment, the shell may be filled with 80 cc - 1,445 cc of the biocompatible filler material.

In one embodiment, the scaffold may have holes or ports formed therein to allow the gel to flow between the interior and exterior regions of the scaffold.

In one embodiment, providing gel outside the scaffold (e.g., between the outer surface of the scaffold and the front wall portion of the shell) is beneficial because the gel is generally soft to the touch.

In one embodiment, the scaffold inhibits the type of gel flow found in prior art devices that tends to pull and distort the shape of the shell. Thus, the scaffold disclosed in the present patent application provides a unique shape maintaining effect.

In one embodiment, an implantable prosthetic device having a scaffold incorporated therein has the appearance of an implant that has been filled with extra gel, but without requiring the extra gel. Rather than using extra gel to provide more form stability, the scaffold disclosed in the present patent application functions as a substitute for the need to use extra gel, which reduces firmness and the overall weight of the implant.

These and other preferred embodiments of the present patent application will be described in more detail below.

### BRIEF DESCRIPTION OF THE DRAWINGS

FIG. 1A is a perspective view of an implantable device including an outer shell, in accordance with one embodiment of the present patent application.

FIG. 1B is a front elevation view of the implantable device shown in FIG. 1A.

FIG. 2 is a cross-sectional view of the implantable device shown in FIGS. 1A and 1B including the outer shell, a scaffold disposed within the outer shell, and a gel contained within the outer shell and engaging a surface of the scaffold, in accordance with one embodiment of the present patent application.

FIG. 3 is a cross-sectional view of the implantable device shown in FIG. 1B including the outer shell, the scaffold, and the silicone gel contained within the outer shell and in contact with at least the surface of the scaffold.

FIG. 4A is a side elevation view of a scaffold for an implantable device. The scaffold having a dome-shaped wall and openings formed in the dome-shaped wall.

FIG. 4B is a perspective view of an underside of the scaffold shown in FIG. 4A.

FIG. 5 is a perspective view of an implantable device including an outer shell, a scaffold disposed within the outer shell, and a silicone gel contained within the outer shell and in contact with at least one surface of the scaffold disposed within the outer shell, in accordance with one embodiment of the present patent application.

FIG. 6 shows the implantable device of FIG. 5 when the device is rotated 90° in the vertical orientation.

FIG. 7 is a schematic, cross-sectional view of an outer shell of an implantable device, in accordance with one embodiment of the present patent application.

FIG. 8 is a schematic, cross-sectional view of a first scaffold having a first outer dimension and a second scaffold having a second outer dimension that is smaller than the first outer dimension, in accordance with one embodiment of the present patent application.

FIG. 9 is a schematic, cross-sectional view of the outer shell of FIG. 7 with the first and second scaffolds of FIG. 8 disposed within the outer shell, in accordance with one embodiment of the present patent application.

FIG. 10 is a schematic, cross-sectional view of an outer shell of an implantable device, in accordance with one embodiment of the present patent application.

FIG. 11 is a schematic, cross-sectional view of a scaffold configured for being disposed within the outer shell of FIG. 10, the scaffold having a dome-shaped wall with a plurality of slits formed in the dome-shaped outer wall.

FIG. 12 is a cross-sectional view of the shell shown in FIG. 10 with the scaffold of FIG. 11 disposed within the outer shell, in accordance with one embodiment of the present patent application.

FIG. 13 is a schematic, cross-sectional view of an outer shell of an implantable device, in accordance with one embodiment of the present patent application.

FIG. 14 is a schematic, cross-sectional view of a scaffold for an implantable device, the scaffold having a dome-shaped wall with an apex region having a first thickness and a radial region having a second thickness that is less than the first thickness, in accordance with one embodiment of the present patent application.

FIG. 15 shows the outer shell of FIG. 13 with the scaffold of FIG. 14 disposed therein, in accordance with one embodiment of the present patent application.

FIG. 16 is a schematic, cross-sectional view of an outer shell of an implantable device, in accordance with one embodiment of the present patent application.

FIG. 17 is a schematic, cross-sectional view of a scaffold having a dome-shaped outer wall with an inner surface that is concave and an outer surface that is asymmetric, in accordance with one embodiment of the present patent application.

FIG. 18 shows the outer shell of FIG. 16 with the scaffold of FIG. 17 contained therein, in accordance with one embodiment of the present patent application.

FIG. 19 is a schematic, cross-sectional view of an outer shell of an implantable device, in accordance with one embodiment of the present patent application.

FIG. 20 is a schematic, cross-sectional view of a scaffold that is configured to be disposed within the outer shell of FIG. 19, the scaffold having a dome-shaped wall with an inner surface including ribs, in accordance with one embodiment of the present patent application.

FIG. 21 shows the outer shell of FIG. 19 with the scaffold of FIG. 20 disposed therein, in accordance with one embodiment of the present patent application.

FIG. 22 is a schematic, cross-sectional view of a shell of an implantable device, in accordance with one embodiment of the present patent application.

FIG. 23 is a schematic, cross-sectional view of a scaffold configured for being disposed within the outer shell of FIG. 22, the scaffold having a dome-shaped wall with a rim that projects inwardly at a lower end of the scaffold to define an open base.

FIG. 24 is a cross-sectional view of the shell shown in FIG. 22 with the scaffold of FIG. 23 disposed within the outer shell, in accordance with one embodiment of the present patent application.

FIG. 25 is a schematic, cross-sectional view of an outer shell of an implantable device, in accordance with one embodiment of the present patent application.

FIG. 26 is a schematic, cross-sectional view of a first scaffold having a first outer dimension and a second scaffold having a second outer dimension that is smaller than the first outer dimension, in accordance with one embodiment of the present patent application.

FIG. 27 is a schematic, cross-sectional view of the outer shell of FIG. 25 with the first and second scaffolds of FIG. 26 disposed within the outer shell, in accordance with one embodiment of the present patent application.

FIG. 28 is a schematic, cross-sectional view of an outer shell of an implantable device, in accordance with one embodiment of the present patent application.

FIG. 29 is a schematic, cross-sectional view of a scaffold that is configured to be disposed within the outer shell of FIG. 28, in accordance with one embodiment of the present patent application.

FIG. 30 shows the outer shell of FIG. 28 with the scaffold of FIG. 29 disposed therein, and struts extending between the scaffold and the shell, in accordance with one embodiment of the present patent application.

FIG. 31 is a schematic, cross-sectional view of an outer shell of an implantable device, in accordance with one embodiment of the present patent application.

FIG. 32 is a schematic, cross-sectional view of a scaffold that is configured to be disposed within the outer shell of FIG. 31, the scaffold having a dome-shaped wall with an outer surface including ribs, in accordance with one embodiment of the present patent application.

FIG. 33 shows the outer shell of FIG. 31 with the scaffold of FIG. 32 disposed therein, and the ribs maintaining spacing between the scaffold and the shell, in accordance with one embodiment of the present patent application.

FIG. 34 is a side elevation view of a scaffold for an implantable device, the scaffold including a dome-shaped wall with a plurality of openings formed in the dome-shaped wall, in accordance with one embodiment of the present patent application.

FIG. 35 shows a side elevation view of an implantable device including an outer shell and the scaffold of FIG. 34 disposed within the outer shell, the implantable device including a silicone gel contained within the outer shell that is contact with at least one surface of the scaffold, in accordance with one embodiment of the present patent application.

FIG. 36 shows the implantable device of FIG. 35 rotated 90 degrees in a vertical orientation.

FIG. 37 is a side elevation view of a scaffold of an implantable device, the scaffold including a dome-shaped wall having a plurality of openings formed therein, in accordance with one embodiment of the present patent application.

FIG. 38 is a side elevation view of an implantable device including an outer shell and the scaffold of FIG. 37 disposed within the outer shell, the implantable device including gel contained within the outer shell and in contact with at least one surface of the scaffold, in accordance with one embodiment of the present patent application.

FIG. 39 shows the implantable device of FIG. 38 after being rotated 90 degrees into a vertical orientation.

FIG. 40 shows a prior art implantable device.

FIG. 41 shows a prior art implantable device.

FIG. 42 shows a prior art implantable device.

FIG. 43 shows a prior art implantable device.

### DETAILED DESCRIPTION OF PREFERRED EMBODIMENTS

In one embodiment, the elements of the implantable prosthetic devices disclosed herein may be defined as set forth below.

Implantable prosthetic device. A mammary implant or tissue expander, which is filled with a biocompatible cohesive filler material such as a gel. An implantable prosthetic device may be pre-filled, filled intraoperatively, or may be filled in situ. Breast implants are typically pre-filled.

Shell. The outer envelope of the implantable prosthetic device, which contains the biocompatible filler material (e.g., a cohesive filler material; silicone gel). The shell is typically made of biocompatible polymers such as silicone, however, other materials may be used. The shell is also referred to as the outer shell.

Apex. The top of the dome of the shell.

Radius. The side region of the implantable prosthetic device where the dome of the shell comes down to intersect with the base of the shell.

Dome. The rounded region of the shell running from the apex to the radius region.

Radial direction. Running in general direction from the apex to the base of the shell and/or running in a plane that is perpendicular to the base.

Circumferential direction. A direction that extends around the sides of an implantable prosthetic device and/or in a plane that is parallel with the base of the shell, such as the radius region of a breast implant or tissue expander.

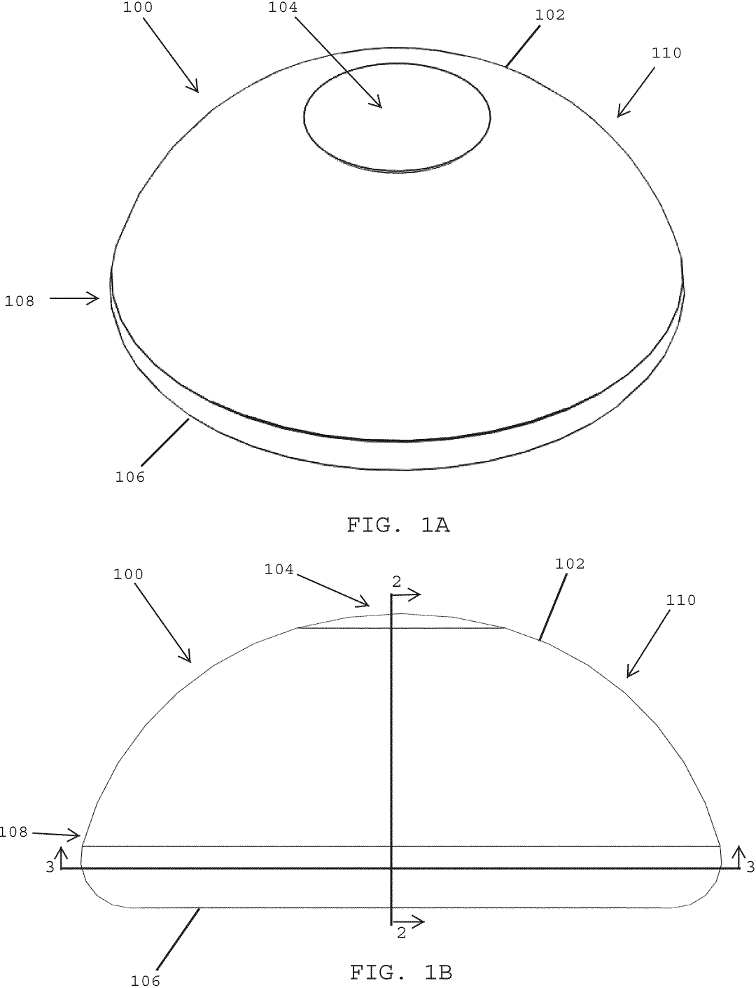

Referring to FIGS. 1A and 1B, in one embodiment, an implantable prosthetic device 100 (e.g., a breast implant) preferably includes a shell 102 having an apex 104, a base 106, a radius 108 that extends around the side of the implantable prosthetic device 100, and a dome 110 that extends from the apex 104 to the radius 108.

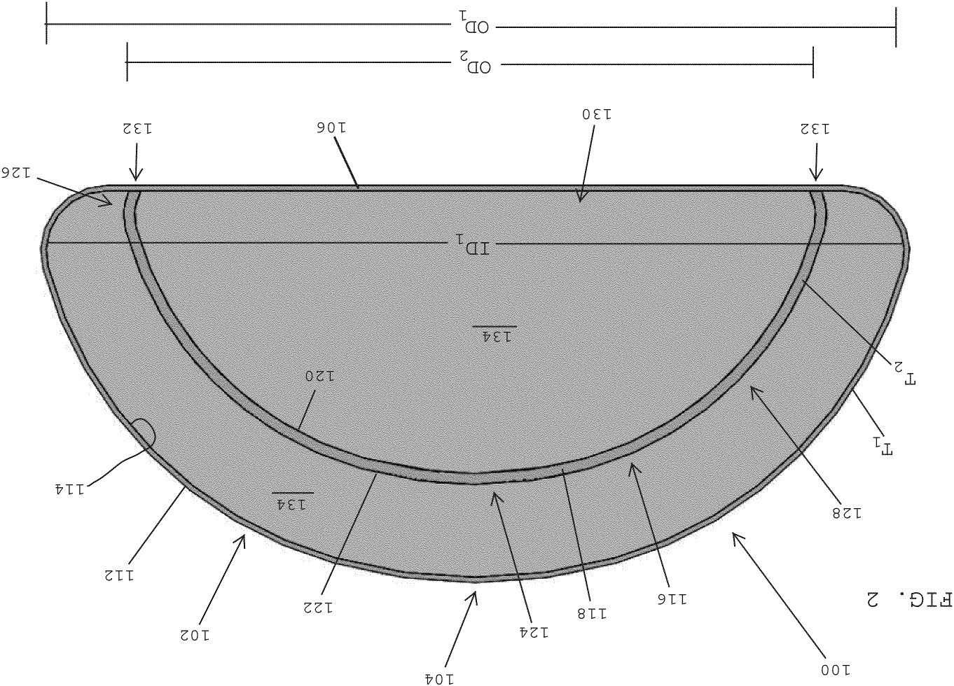

Referring to FIG. 2, in one embodiment, the shell 102 of the implantable prosthetic device 100 preferably has an outer surface 112 and an inner surface 114. In one embodiment, the outer surface 112 of the shell 102 may have a section that is convexly curved and the inner surface 114 of the shell 102 may have a section that includes a concave curve. The outer surface of the shell may be smooth or may have a textured surface. The implantable device may include suture tabs that are attached to the shell for securing the shell to tissue using sutures.

In one embodiment, the shell 102 may be formed by depositing (e.g., spraying, dipping) a biocompatible curable material over a convexly curved outer surface of a mandrel. In one embodiment, the curable material that is applied over the convexly curved outer surface of the mandrel may be a curable silicone material. In one embodiment, the curable silicone material may be sprayed over the outer surface of a mandrel, whereupon the curable material flows over the convexly curved surface. In one embodiment, the curable silicone material may be applied by dipping the mandrel in a curable silicone solution. In one embodiment, the shell may have multiple layers that are built up over the convexly curved outer surface of the mandrel using multiple spraying and/or dipping steps, whereby multiple layers of the curable material are deposited for increasing the wall thickness of shell.

In one embodiment, the shell 102 may be made using one or more of the systems, devices and methods disclosed in U.S. Pat. No. 4,472,226 to Redinger et al., U.S. Pat. Application Publication No. US 2014/0088703 to Schuessler, or U.S. Pat. No. 10,898,313, the disclosures of which are hereby incorporated by reference herein.

In one embodiment, the shell 102 has an interior volume or interior chamber that may be filled with a biocompatible filler material. In one embodiment, the biocompatible filler material may include a gel, saline, water, air, a biocompatible gas (e.g., nitrogen), or combinations thereof. In one preferred embodiment, the biocompatible filler material that fills the interior volume of the shell 102 is a silicone gel, which may be uncured, partially cured, or fully cured.

In one embodiment, the implantable prosthetic device 100 preferably includes a scaffold 116 (e.g., a buttress; a support; a brace) that is disposed within the interior volume of the shell 102. The scaffold 116 may have the shape of a hollow hemisphere. In one embodiment, the scaffold 116 may include a dome-shaped wall 118 having a concave bottom surface 120 that faces toward the base 106 of the shell 102 and a convexly curved top surface 122 that faces toward the apex 104 of the shell 102.

In one embodiment, the dome-shaped wall 118 of the scaffold 116 preferably includes an apex 124, a radius 126, and a dome region 128 that extends between the apex 124 and the radius 126.

In one embodiment, the scaffold 116 preferably has an open base 130 that is positioned over the base 106 of the outer shell 102. In one embodiment, the open base 130 of the scaffold 116 is surrounded by a free edge 132 of the outer wall 118 of the scaffold 116. The free edge 132 of the scaffold may have a circular shape. In one embodiment, the base of the scaffold may include a rim that projects inwardly from an outer perimeter. The rim may have an inner edge that surrounds an opening formed in the base of the scaffold. In one embodiment, the scaffold may include a wall having a curved region that extends between the radius or side of the scaffold and the rim that projects inwardly at the base of the scaffold.

In one embodiment, the scaffold 116 may have the appearance of a hollow sphere that has been cut in half by a horizontal plane to form a hollow hemisphere having the open base 130 that is surrounded by the circular, free edge of the dome-shaped wall 118 of the scaffold 116. In one embodiment, the scaffold 116 may be made of the same material that is used to make the shell 102. In one embodiment, the scaffold may be made of a material that is different than the material that is used to make the shell.

In one embodiment, the wall of the shell 102 has a first thickness T₁ that is smaller than the second thickness T₂ of the wall 118 of the scaffold 116. In one embodiment, the thickness of the wall 118 of the scaffold 116 may vary between the apex 124 of the scaffold and the radius 126 of the scaffold.

In one embodiment, a biocompatible filler material 134, such as a silicone gel, may be disposed within the interior volume of the shell 102 of the implantable prosthetic device 100. The biocompatible filler material 134 preferably fills the interior volume of the shell 102 and surrounds the scaffold 116 that is contained within the shell 102. In one embodiment, the biocompatible filler material 134 is adhered to one or more surfaces 120, 122 of the wall 118 of the scaffold 116.

In one embodiment, the scaffold 116 preferably overlies an inner surface of the base 106 of the shell 102. In one embodiment, the scaffold 116 may be centered over the base 106 of the shell 102.

In one embodiment, the circular, free edge 132 of the scaffold 116 is preferably juxtaposed with the inner surface of the base 106 of the shell 102. In one embodiment, the circular, free edge 132 of the scaffold 116 may be adhered and/or secured to the inner surface of the base 106 of the scaffold 116. In one embodiment, the circular, free edge 132 of the scaffold 116 is not adhered to the inner surface of the base 106 of the scaffold 116.

In one embodiment, the shell 102 has a first outer diameter OD₁ that is larger than the second outer diameter OD₂ of the scaffold 116. In one embodiment, the shell 102 has a first inner diameter ID₁ that is larger than the second outer diameter OD₂ of the scaffold 116.

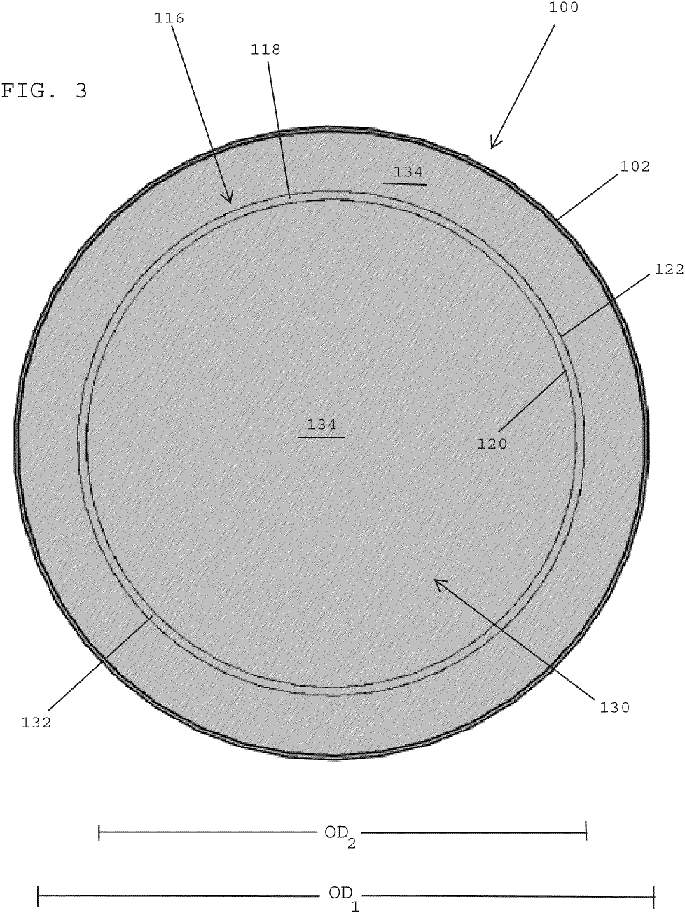

Referring to FIG. 3, in one embodiment, the scaffold 116 is disposed within the shell 102 of the implantable device 100. The dome-shaped wall 118 of the scaffold 116 has the circular, free edge 132 that surrounds the open base 130 of the scaffold 116. The circular, free edge 132 is spaced inwardly and away from the outer perimeter of the shell 102.

In one embodiment, the circular, free edge 132 of the dome-shaped wall 118 of the scaffold 116 may be centered over the base 106 (FIG. 2) of the shell 102 of the implantable device 100. The shell 102 has a first outer diameter OD₁ that is larger than the second outer diameter OD₁ of the scaffold 116.

In one embodiment, the biocompatible filler material 134 preferably fills the interior volume of the shell 102. In one embodiment, the biocompatible filler material is in contact with one or more of the major surfaces 120, 122 of the dome-shaped wall 118 of the scaffold 116. In one embodiment, the biocompatible filler material is adherent to one or more of the major surfaces of the scaffold. In one embodiment, the biocompatible filler material completely fills the interior volume of the shell and the interior volume of the scaffold that is disposed inside the shell. In one embodiment, the biocompatible filler material is disposed between the outer surface 122 of the scaffold 118 and the inner surface of the wall of the shell 102.

Referring to FIGS. 4A and 4B, in one embodiment, a scaffold 216 for an implantable prosthetic device (e.g., a breast implant) preferably includes an outer wall 218 having a dome-shape with an open base 230. The scaffold 216 may be made of a biocompatible material, such as a biocompatible polymer (e.g., silicone). The scaffold 216 may have the shape of hollow hemisphere having a lower end with an open base 230 that is surrounded by a circular, free edge 232 of the outer wall 218. The circular, free edge 232 preferably extends around the outer perimeter of the open base 230 of the scaffold 216.

In one embodiment, the dome-shaped wall 218 of the scaffold 216 preferably includes a plurality of openings 236 formed therein that extend between the inner surface 220 and the outer surface 222 of the outer wall 218 of the scaffold 216. The openings 236 may have various geometric shapes including circles. In other embodiments, the openings may be elongated and/or may include slits that are formed in the outer wall 218 and that extend from the outer surface to the inner surface of the dome-shaped wall 218. The openings 236 may have other geometric shapes, including, but not limited to, squares, rectangles, polygons, and triangles.

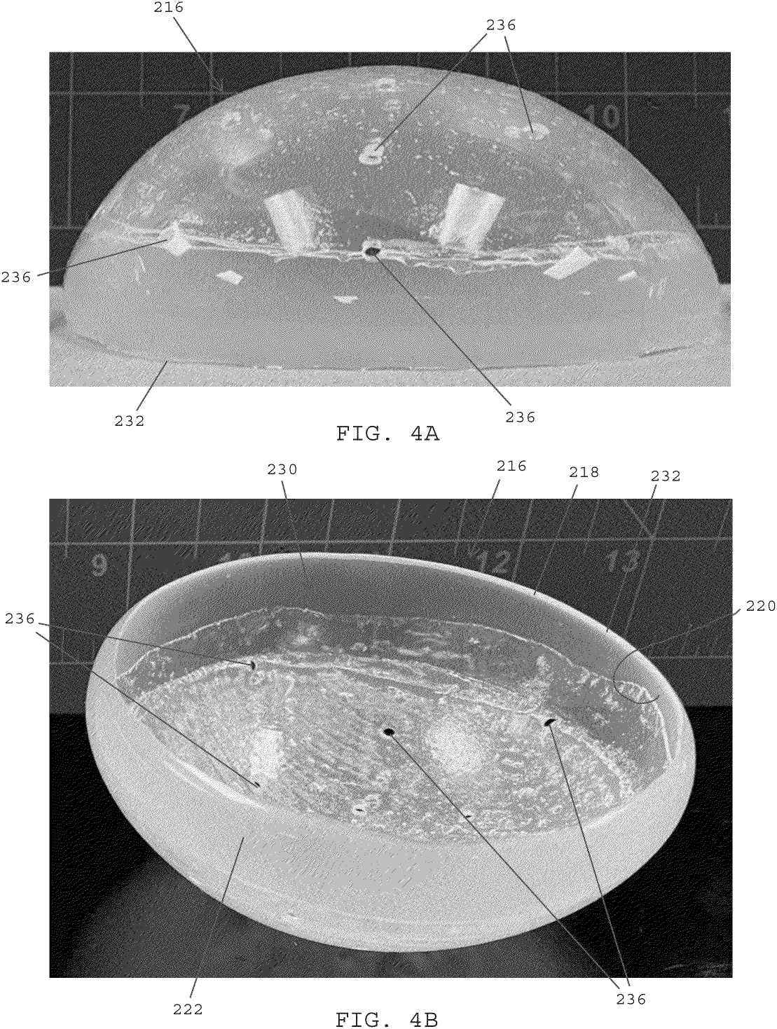

Referring to FIG. 5, in one embodiment, the scaffold 216 shown and described above in FIGS. 4A and 4B may be disposed within a shell 202 of an implantable prosthetic device 200. The open base 230 (FIG. 4B) of the scaffold 216 is preferably juxtaposed with the base 206 (i.e., a rear wall) of the shell 202. In one embodiment, the circular, free edge 232 (FIG. 4B) of the dome-shaped wall 218 of the scaffold 216 preferably abuts against and/or is juxtaposed with an inner surface of the base 206 of the shell 202.

In one embodiment, the shell 202 is preferably filled with a biocompatible filler material (e.g., a silicone gel), which completely fills the interior volume of the shell 202 and is adherent to one or more major surfaces of the dome-shaped wall 218 of the scaffold 216. In one embodiment, the biocompatible filler material that fills the interior volume of the shell 202 may pass through the one or more of the openings 236 (FIG. 4B) formed in the dome-shaped wall 218 of the scaffold 216.

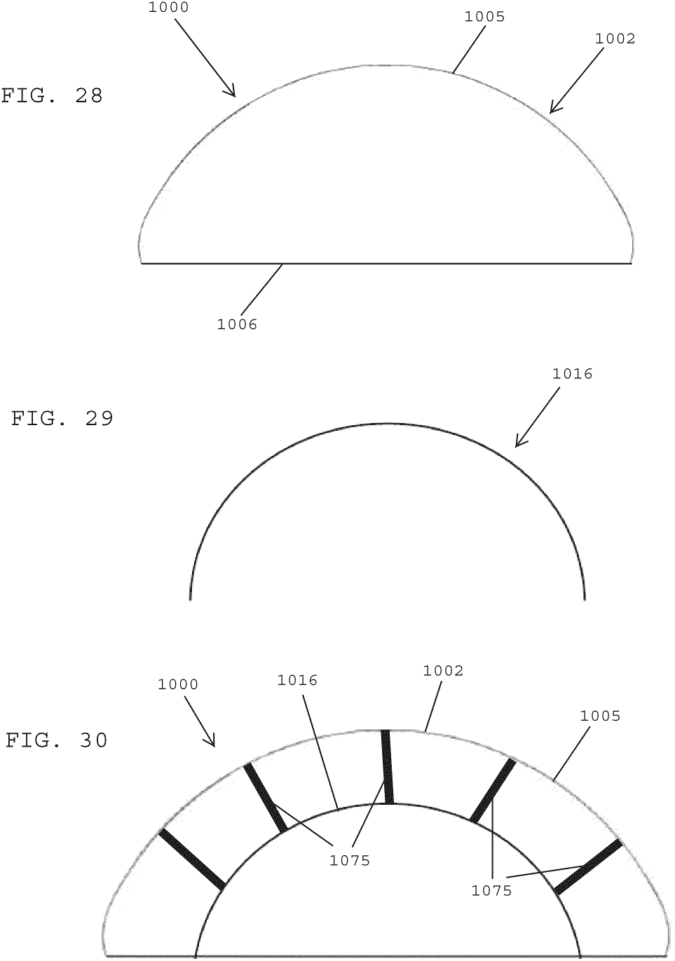

In FIG. 5, the shell 202 of the implantable prosthetic device 200 is in a horizontal orientation with the base 206 of the shell 202 overlying a horizontal surface. FIG. 6 shows the implantable prosthetic device 200 of FIG. 5 after the shell 202 has been rotated 90 degrees in a clockwise direction relative to the horizontal orientation shown in FIG. 5. In FIG. 6, the shell 202 may be described as being in a vertical orientation. The scaffold 216 that is disposed inside the shell 202 of the implantable device 200 preferably provides an internal reinforcement for the shell that maintains the shape and form stability of the shell. The base 206 of the shell, reinforced by the scaffold 216, extends along a vertical axis and the shell does not lose its shape as occurs with the prior art implants shown in FIGS. 28-31 of the present patent application.

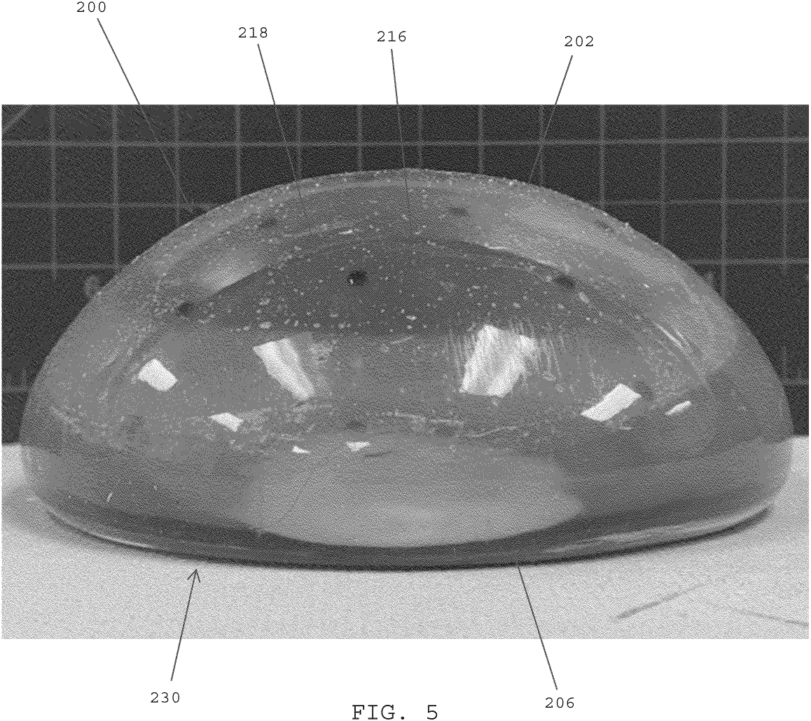

Referring to FIG. 7, in one embodiment, an implantable prosthetic device 300 (e.g., a breast implant) preferably includes a shell 302 having a front wall 305 and a rear wall 306 (i.e., a base). The front wall 305 of the shell 302 may include an apex 304, a radius 308, and a dome 310 that extends between the apex 304 and the radius 308.

Referring to FIG. 8, in one embodiment, the implantable prosthetic device preferably includes a first scaffold 316A having a first dimension and a second scaffold 316B having a second dimension that is smaller than the first dimension of the first scaffold 316A.

In one embodiment, the second scaffold 316B is configured for nesting inside the first scaffold 316A.

In one embodiment, the first scaffold 316A defines a first height H₁ and an outer diameter OD₃.

In one embodiment, the second scaffold 316B defines a second height Hz and an outer diameter OD₂. In one embodiment, the first height H₁ of the first scaffold 316A is greater than the second height Hz of the second scaffold 316B. Similarly, the outer diameter OD₃ of the first scaffold 316A is greater than the outer diameter OD₄ of the second scaffold 316B.

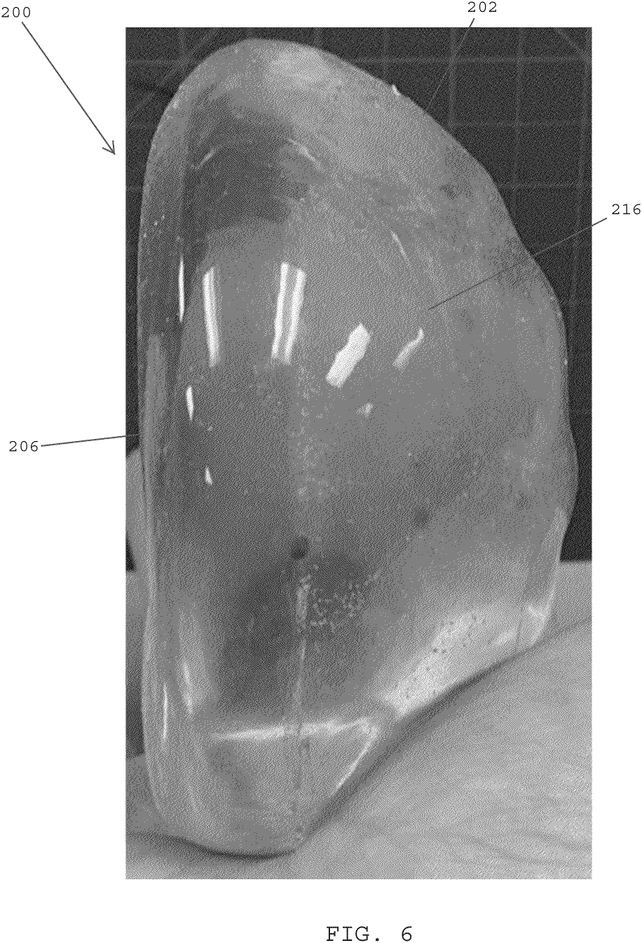

Referring to FIGS. 8 and 9, in one embodiment, the first and second scaffolds 316A, 316B are disposed within the interior volume of the shell 302 of the implantable device 300. In one embodiment, the second scaffold 316B is nested within the first scaffold 316A. The first and second scaffolds preferably have open bases so that circular, free edges 332A, 332B of the respective first and second scaffolds are juxtaposed with the inner surface of the rear wall 306 of the shell 302 of the implantable device 300.

In one embodiment, the second scaffold 316B may have a wall thickness that is greater than the wall thickness of the first scaffold 316A. In one embodiment, the first scaffold 316A may have a wall thickness that is greater than the wall thickness of the shell 302 of the implantable device 300. In one embodiment, the first and second scaffolds 316A, 316B may have respective wall thicknesses that are equal and/or similar to one another.

In one embodiment, an implantable prosthetic device may include three or more scaffolds that are preferably nested within one another and that are disposed inside a shell of the implantable prosthetic device. Each of the three or more scaffolds preferably has a different size.

In one embodiment, the shell 302 of the implantable prosthetic device 300 is preferably filled with a gel, such as a silicone gel. The silicone gel preferably engages one or more surfaces of the first and second scaffolds 316A, 316B.

Referring to FIG. 10, in one embodiment, an implantable prosthetic device 400 preferably includes a shell 402 having a front wall 405 and a rear wall 406.

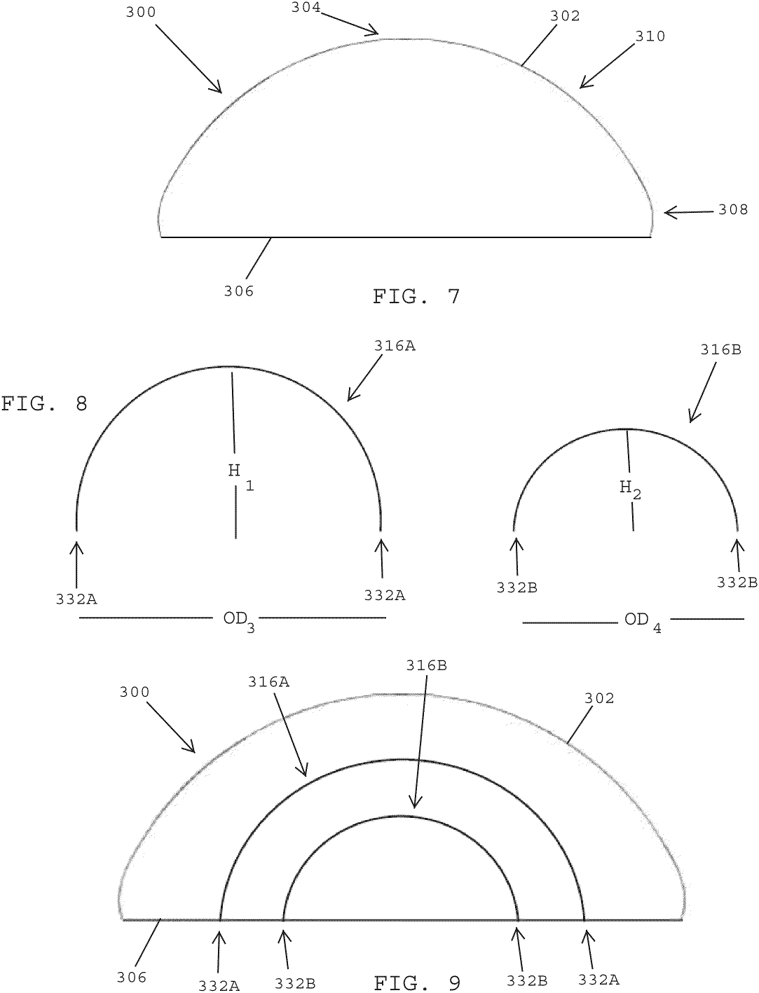

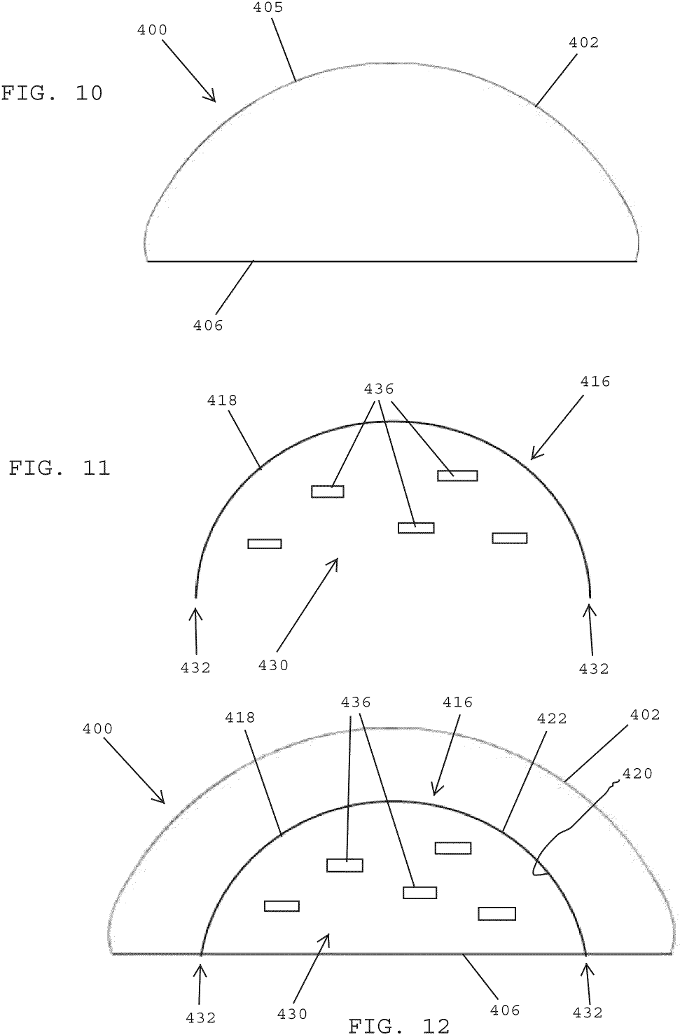

Referring to FIG. 11, in one embodiment, a scaffold 416 is configured for being disposed within the interior volume of the shell 402 of the implantable prosthetic device 400 shown in FIG. 10. In one embodiment, the scaffold 416 preferably includes a wall 418 having a dome shape and an open base 430 surrounded by a circular, free edge 432 of the dome-shaped wall 418. In one embodiment, the dome-shaped wall 418 of the scaffold 416 has a plurality of slits 436 (e.g., elongated openings) formed therein that extend between an inner surface 420 and an outer surface 422 of the dome-shaped wall 418 of the scaffold 416.

Referring to FIG. 12, in one embodiment, the scaffold 416 is disposed within the shell 402 so that the circular, free edge 432 of the scaffold that surrounds the open base 430 of the scaffold 416 is juxtaposed with an inner surface of the rear wall 406 of the shell 402.

In one embodiment, the scaffold is centered over the rear wall 406 of the shell 402. In one embodiment, the circular, free edge of the scaffold may be adhered to the rear wall 406 of the shell 402.

In one embodiment, the interior volume of the shell of the implantable prosthetic device 400 is preferably filled with a biocompatible filler material, such as a silicone gel, which is adhered to one or more major surfaces 420, 422 of the wall 418 of the scaffold 416.

Referring to FIG. 13, in one embodiment, an implantable prosthetic device 500 preferably includes a shell 502 (e.g., a silicone shell) having a front wall 505 and a rear wall 506 that is adapted to receive a scaffold (e.g., a reinforcing support; a buttress).

Referring to FIG. 14, in one embodiment, a scaffold 516 adapted to be disposed within the interior volume of the shell 502 of FIG. 13. The scaffold 516 preferably includes a wall 518 having a dome-shape with an open base 530 that is surrounded by a circular, free edge 532 of the wall 518 of the scaffold 516. In one embodiment, the dome-shaped wall 518 of the scaffold 516 preferably includes an apex 524, a radius 526, and a dome-shaped region 528 that extends between the apex 524 and the radius 526. In one embodiment, the wall 518 of the scaffold 516 is thicker in the apex region 524 thereof and thinner in the radius region 526 thereof.

Referring to FIG. 15, in one embodiment, the scaffold 516 is disposed within the interior volume of the shell 502 of the implantable prosthetic device 500. The circular, free edge 532 that surrounds the open base 530 of the scaffold 516 is preferably juxtaposed with the inner surface of the rear wall 506 of the shell 502. In one embodiment, the circular, free edge 532 of the scaffold 516 may be adhered and/or connected with the rear wall 506 of the shell 502.

In one embodiment, the shell 502 is preferably filled with a biocompatible filler material, such as a silicone gel. The silicone gel desirably adheres to one or more of the major surfaces 520, 522 of the wall 518 of the scaffold 516.

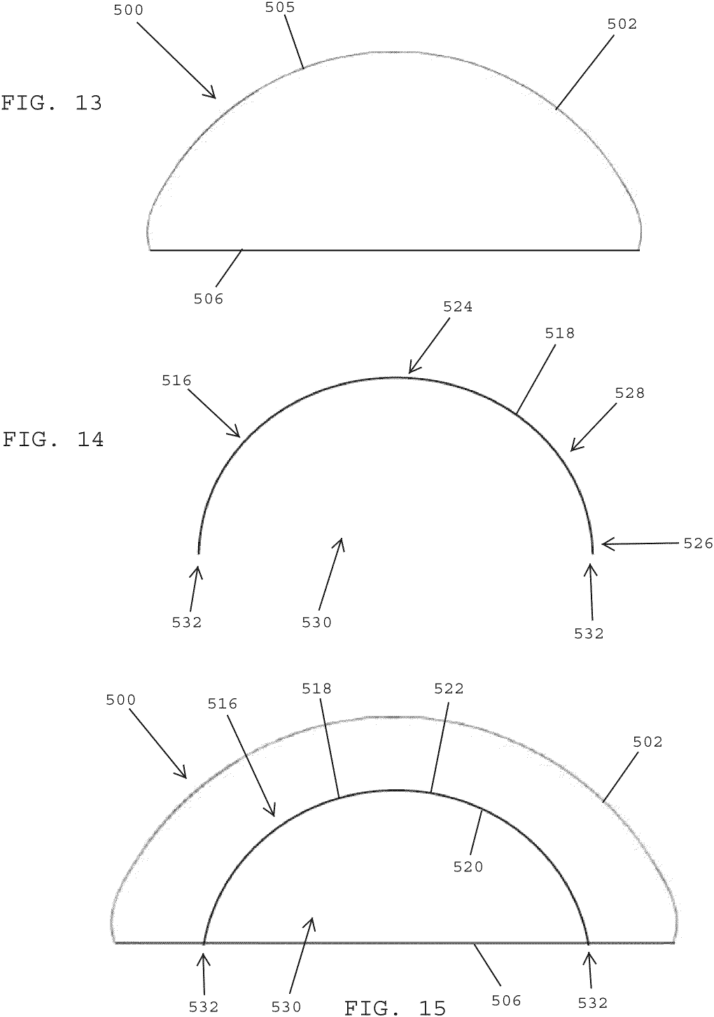

Referring to FIG. 16, in one embodiment, an implantable prosthetic device 600 preferably includes a shell 602 having a front wall 605 and a rear wall 606. In one embodiment, the shell 602 is configured for receiving a scaffold that provides form stability to the shell.

Referring to FIG. 17, in one embodiment, a scaffold 616 includes a curved wall 618 having a dome shape. The scaffold 616 preferably has a hollow, hemispheric shape with an open base 630 that is surrounded by a circular, free edge 632 of the curved wall 618. In one embodiment, the curvature of the curved wall 618 of the scaffold 616 does not mirror the geometric shape or curvature of the front wall 605 of the shell 602.

In one embodiment, the curvature of the wall 618 of the scaffold 616 is greater than the curvature of the front wall 605 of the outer shell 602 of the implantable prosthetic device 600. Thus, the curvature and/or geometry of the wall 618 of the scaffold 616 neither matches nor mirrors the curvature and/or geometry of the front wall 605 of the shell 602 of the implantable prosthetic device 600.

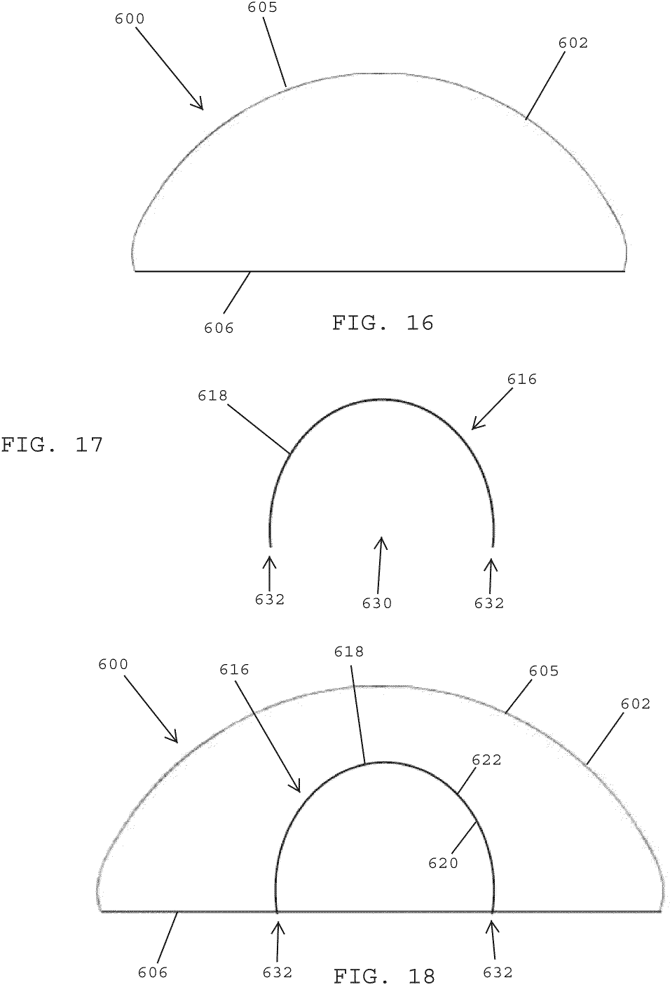

Referring to FIG. 18, in one embodiment, the scaffold 616 is disposed within the shell 602 of the implantable prosthetic device 600. The circular, free edge 632 of the wall 618 of the scaffold 616 is preferably juxtaposed with the rear wall 606 of the outer shell 602. The curvature of the wall 618 of the scaffold 616 does not match the curvature of the front wall 605 of the outer shell 602.

In one embodiment, the scaffold 616 is preferably centered over the rear wall 632 of the outer shell 602. In one embodiment, the circular, free edge 632 of the scaffold 616 may be secured and/or adhered to the inner surface of the rear wall 606 of the shell 602.

In one embodiment, the interior volume of the shell 602 of the implantable device 600 is preferably filled with a biocompatible filler material (e.g., a silicone gel) that engages one or more of the major surfaces 620, 622 of the wall 618 of the scaffold 616.

Referring to FIG. 19, in one embodiment, an implantable prosthetic device 700 preferably includes a shell 702 having a front wall 705 and a rear wall 706.

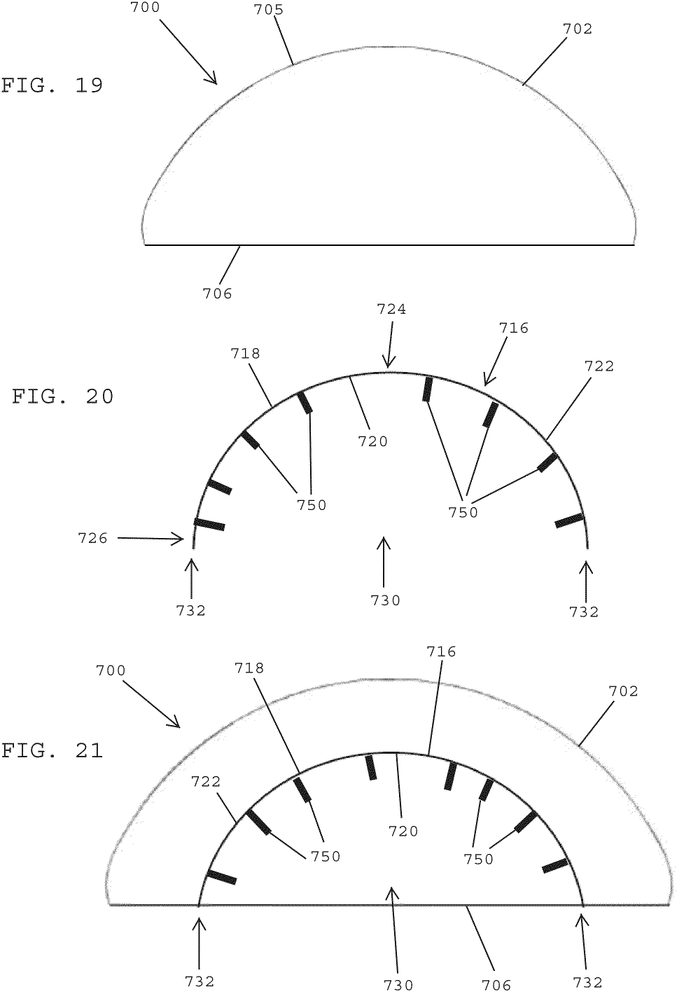

Referring to FIG. 20, in one embodiment, a scaffold 716 is preferably configured for being disposed within the shell 702 of the implantable prosthetic device 700 shown and described above in FIG. 19. In one embodiment, the scaffold 716 preferably includes a wall 718 having a dome shape with a concave inner surface 720 and a convexly curved outer surface 722. In one embodiment, the scaffold 716 desirably includes an open base 730 that is surrounded by a circular, free edge 732 of the wall 718 of the scaffold 716.

In one embodiment, the scaffold 716 preferably includes one or more ribs 750 that are connected with the inner surface 720 of the scaffold 716 and that project inwardly from the inner surface 720 of the wall 718 of the scaffold. The ribs 750 may be integrally formed with the wall 718 of the scaffold 716. The ribs 750 may be formed using one or more of the systems, devices and methods disclosed in commonly assigned U.S. Pat. No. 10,898,313, the disclosure of which is hereby incorporated by reference herein.

In one embodiment, the one or more ribs 750 preferably include a plurality of ribs that project inwardly from the inner surface of the wall 718 of the scaffold 716. The ribs 750 may be spaced from one another over the inner surface 720 of the wall 718 for enhancing the structural stability of the scaffold 716. In one embodiment, the ribs 750 may extend in radial directions from the apex 724 of the scaffold to the base 730 of the scaffold. In one embodiment, the ribs 750 may extend in circumferential directions around the radius 726 of the scaffold 716.

Referring to FIG. 21, in one embodiment, the scaffold 716 having ribs 750 is preferably disposed within the shell 702 of the implantable prosthetic device 700. In one embodiment, the ribs 750 of the scaffold 716 desirably project from the inner surface of the wall 718 of the scaffold 716 and toward the rear wall 706 of the outer shell 702.

In one embodiment, the circular, free edge 732 of the wall 718 of the scaffold 716 that surrounds the open base 730 is preferably juxtaposed with the rear wall 706 of the outer shell 702. The circular, free edge 732 may be centered over the rear wall 706 of the outer shell 702. In one embodiment, the circular, free edge may be adhered to the rear wall 706 of the shell 702.

In one embodiment, the shell 702 of the implantable device 700 is desirably filled with a biocompatible filler material (e.g., a silicone gel) that completely fills an interior chamber of the shell 702. In one embodiment, the biocompatible filler material is adhered to one or more of the inner and outer surfaces 720, 722 of the curved wall 718 of the scaffold 716.

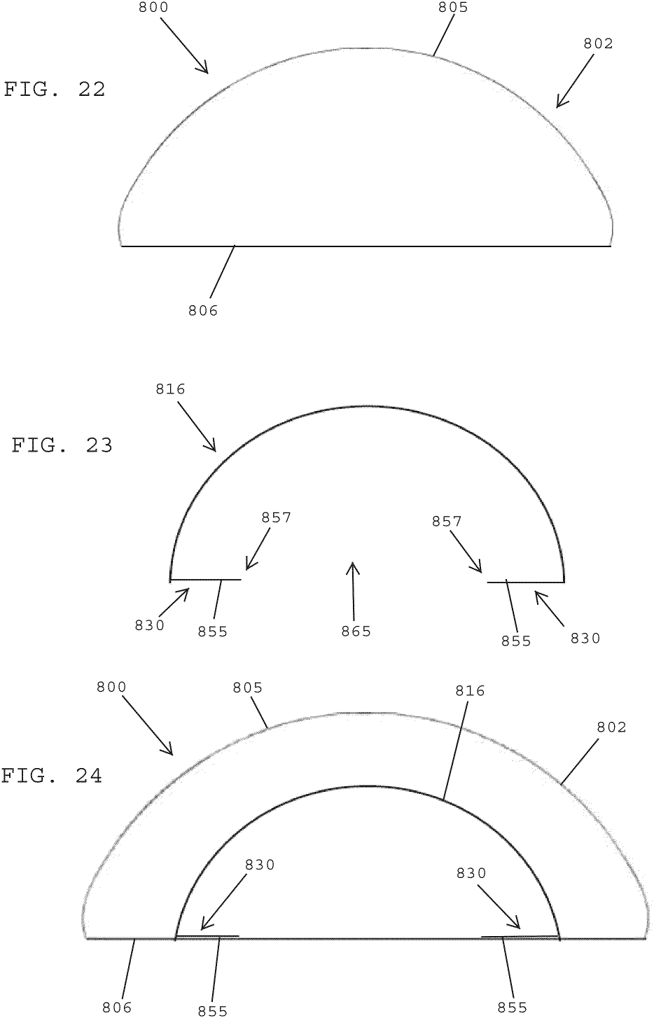

Referring to FIG. 22, in one embodiment, an implantable device 800 preferably includes a shell 802 having a front wall 805 and a base 806, which may also be referred to as a rear wall.

Referring to FIG. 23, in one embodiment, a scaffold 816 has a base 830. A portion of the base 830 may include a rim 855 that projects inwardly at the lower end of the scaffold 816. The rim 855 preferably has a free edge 857 that extends around an opening 865 formed in the base 830. The free edge 857 of the rim 855 may define the size of the opening 865 that is formed in the base 830.

Referring to FIG. 24, in one embodiment, the scaffold 816 may be positioned inside the shell 802 of the implantable device 800 so that the rim 855 at the base 830 of the scaffold 816 engages and/or is juxtaposed with the inner surface of the base 806 of the shell 802. The scaffold 816 preferably has an outer dimension that is smaller than an inner dimension of the front wall 805 of the shell 802 so that the curved wall of the scaffold 816 is spaced away from the curved front wall 805 of the shell 802.

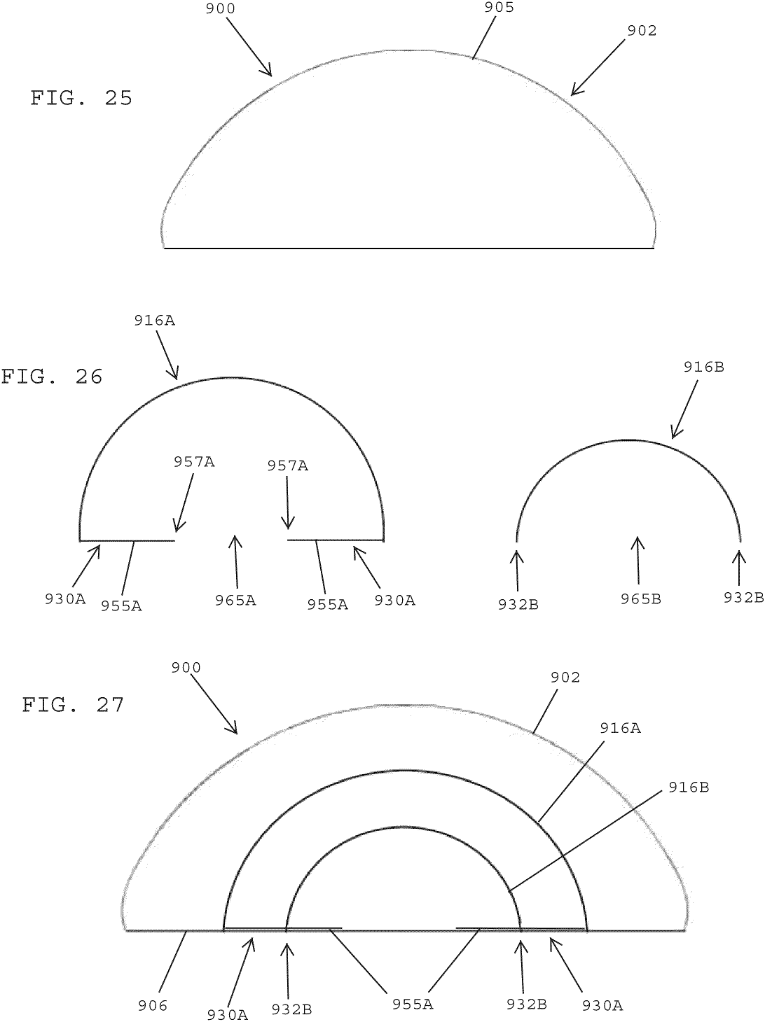

Referring to FIG. 25, in one embodiment, an implantable prosthetic device 900 (e.g., a breast implant) preferably includes a shell 902 having a front wall 905 and a rear wall 906 (i.e., a base).

Referring to FIG. 26, in one embodiment, the implantable prosthetic device preferably includes a first scaffold 916A having a first dimension and a second scaffold 916B having a second dimension that is smaller than the first dimension of the first scaffold 916A.

In one embodiment, the second scaffold 916B is configured for nesting inside the first scaffold 316A.

In one embodiment, the first scaffold 916A has a base 930A with a rim 955A that extends inwardly at the base of the first scaffold. The rim 955A has a free edge 957A that extends around an opening 965A formed in the base 930A. The free edge 957A preferably defines the size of the opening 965A formed in the base 930A of the first scaffold 916A. In one embodiment, the second scaffold 916B has a circular, free edge 932B that defines the size of the opening 965B in the open base 930B of the second scaffold 916B. The second scaffold 916B may be passed through the opening in the opening 965A in the base 930A of the first scaffold 916A for nesting the second scaffold inside the first scaffold. In one embodiment, due to the presence of the inwardly extending rim 955A of the first scaffold 916A, the size of the opening 95B in the open base 930B of the second scaffold 916B is larger than the size of the opening 965A in the base 930A of the first scaffold 916A.

Referring to FIGS. 26 and 27, in one embodiment, the first and second scaffolds 916A, 916B (e.g., nested) may be disposed within the interior volume of the shell 902 of the implantable device 900. In one embodiment, the second scaffold 916B is nested within the first scaffold 916A. The circular, free edge 932B of the second scaffold 916B may be juxtaposed with an inner surface of the inwardly extending rim 955A of the first scaffold 916A, and the outer surface of the inwardly extending rim 955A of the first scaffold 916A may be juxtaposed with the inner surface of the base 906 of the shell 902 of the implantable device 900. In one embodiment, the rim 955A may be adhered to the inner surface of the base 906 of the shell 902. The shell of the implant device 900 may be fille with a biocompatible filler material such as a cohesive filler material or a silicone gel.

Referring to FIG. 28, in one embodiment, an implantable prosthetic device 1000 (e.g., a breast implant) preferably includes a shell 1002 having a front wall 1005 and a rear wall 1006 (i.e., a base).

Referring to FIGS. 29 and 30, in one embodiment, the implantable prosthetic device 1000 preferably includes a scaffold 1016 that is adapted to be disposed within an interior chamber of the shell 1002 (FIG. 28). The scaffold 1016 has an outer radius or dimension that is smaller than an inner radius or dimension of the front wall 1005 of the shell 1002. In one embodiment, the implantable prosthetic device 1000 preferably includes a plurality of struts 1075 (e.g., standoffs) that extend between the scaffold 1016 and the front wall 1005 of the shell 1002. In one embodiment, the struts 1075 preferably maintain spacing and/or standoff between the curved wall of the scaffold 1016 and the front wall 1005 of the shell 1002.

FIG. 30 is a schematic representation of an implant device having struts 1075 for providing spacing and/or standoff between the scaffold 1016 and the front wall 1005 of the shell 1002. The number of struts 1075 extending between the scaffold and the front wall of the shell may be varied. For example, in one embodiment, an implant device may have between two (2) to five struts. In one embodiment, an implant device may have 10, 15, 20, 25, 30, or more struts extending between the scaffold and the front wall of the shell 1002.

Referring to FIG. 31, in one embodiment, an implantable prosthetic device 1100 preferably includes a shell 1102 having a front wall 1105 and a rear wall 1106.

Referring to FIG. 32, in one embodiment, a scaffold 1116 is preferably configured for being disposed within the shell 1102 of the implantable prosthetic device 1100 (FIG. 31). In one embodiment, the scaffold 1116 preferably includes a wall 1118 having a concave shape with a concave inner surface 1120 and a convexly curved outer surface 1122. In one embodiment, the scaffold 1116 desirably includes an open base 1130 that is surrounded by a circular, free edge 1132 of the wall 1118 of the scaffold 1116.

In one embodiment, the scaffold 1116 preferably includes one or more ribs 1150 that are connected with the outer surface 1122 of the scaffold 1116 and that project outwardly from the outer surface 1122 of the wall 1118 of the scaffold. The ribs 1150 may be integrally formed with the wall 1118 of the scaffold 1116. The ribs 1150 may be formed using one or more of the systems, devices and methods disclosed in commonly assigned U.S. Pat. No. 10,898,313, the disclosure of which is hereby incorporated by reference herein. The length and width of the ribs may be modified from what is shown schematically in FIG. 32. The total number of ribs may be modified from what is shown schematically in FIG. 32.

In one embodiment, the one or more ribs 1150 preferably include a plurality of ribs that project outwardly from the outer surface 1122 of the wall 1118 of the scaffold 1116. The ribs 1150 may be spaced from one another over the outer surface 1122 of the wall 1118 for enhancing the structural stability of the scaffold 1116. In one embodiment, the ribs 1150 may extend in radial directions.

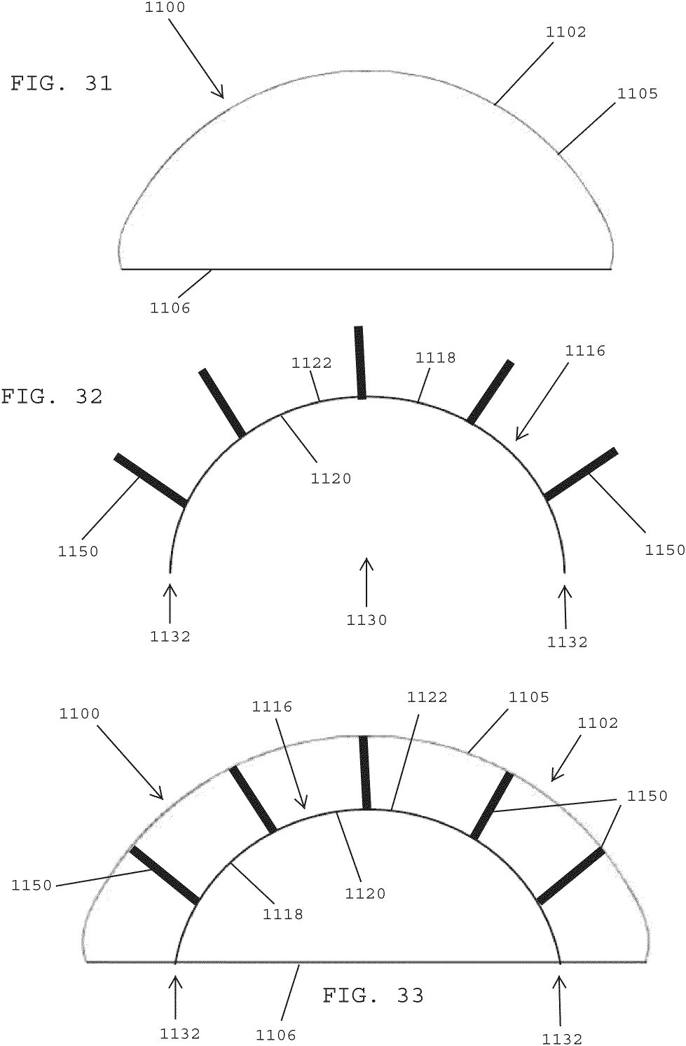

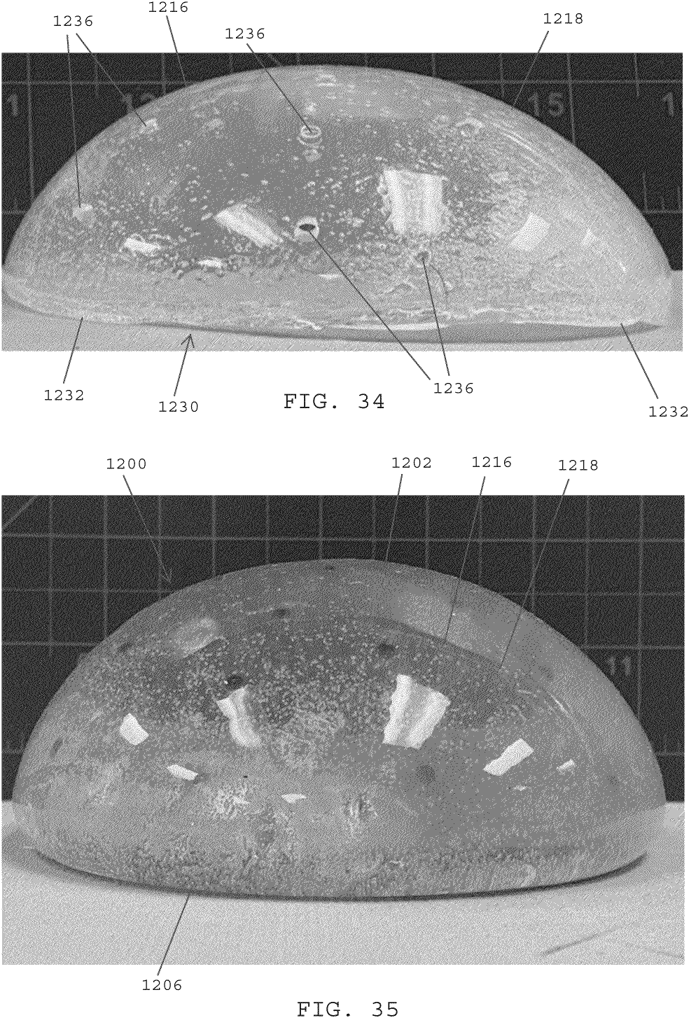

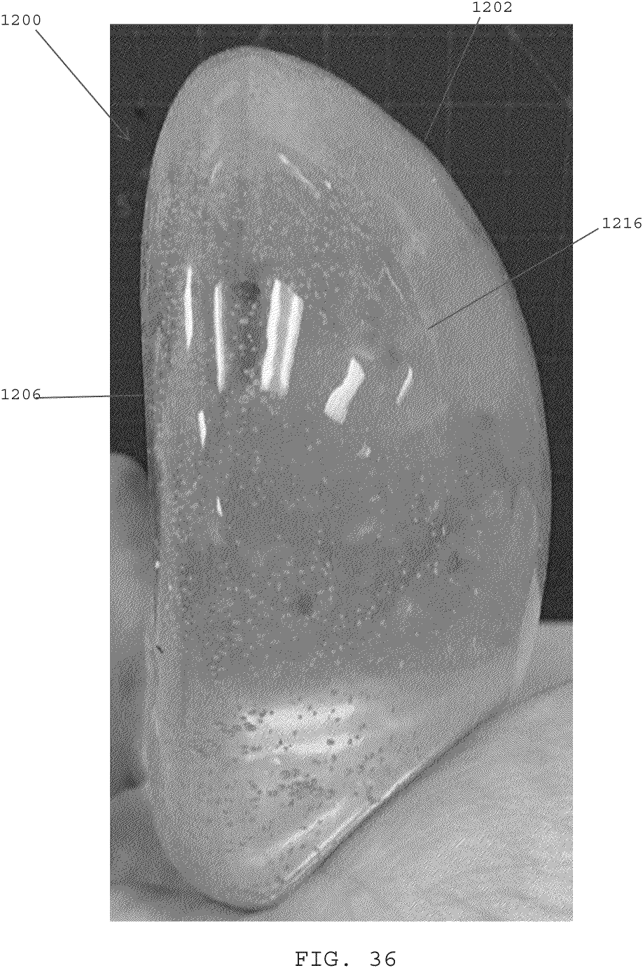

Referring to FIG. 33, in one embodiment, the scaffold 1116 having ribs 1150 may be disposed within the shell 1102 of the implantable prosthetic device 1100. In one embodiment, the ribs 1150 maintain spacing and/or provide standoff between the wall 1118 of the scaffold 1116 and the front wall 1105 of the shell 1102.

In one embodiment, the circular, free edge 1132 of the wall 1118 of the scaffold 1116 is preferably juxtaposed with the base 1106 (i.e., rear wall) of the outer shell 1102. The circular, free edge 1132 may be centered over the base 1106 of the outer shell 1102. In one embodiment, the circular, free edge may be adhered to the base 1106 of the shell 1102.

In one embodiment, the shell 1102 of the implantable device 1100 is desirably filled with a biocompatible filler material (e.g., a cohesive filler material; a silicone gel) that fills an interior chamber of the shell 1102. In one embodiment, the biocompatible filler material is adhered to one or more of the inner and outer surfaces 1120, 1122 of the curved wall 1118 of the scaffold 1116.

Referring to FIG. 34, in one embodiment, a scaffold 1216 for an implantable prosthetic device (e.g., a breast implant) preferably includes a curved wall 1218 having an open base 1230 that is surrounded by a circular, free edge 1232. In one embodiment, the scaffold 1216 may have the shape of a hollow hemisphere.

In one embodiment, the scaffold 1216 may be made of a biocompatible polymer such as a cured silicone gel.

In one embodiment, the scaffold 1216 preferably includes a plurality of openings 1236 formed in the outer wall 1218 thereof that extend from an outer surface to an inner surface of the wall 1218 of the scaffold.

Referring to FIG. 35, in one embodiment, the scaffold 1216 is preferably disposed within a shell 1202 of an implantable prosthetic device 1200 so that the circular, free edge 1232 (FIG. 34) of the scaffold 1216 that surrounds the open base 1230 is juxtaposed with a rear wall 1206 of the shell 1202 of the implantable device 1200. In one embodiment, the scaffold 1216 may be centered over the rear wall 1206 of the shell 1202. The scaffold may be attached to the base of the shell.

In one embodiment, the implantable prosthetic device 1200 includes a biocompatible filler material (e.g., a silicone gel) that fills the interior chamber of the shell 1202 and that is adherent to at least one of the inner and outer surfaces of the curved wall 1218 of the scaffold 1216,

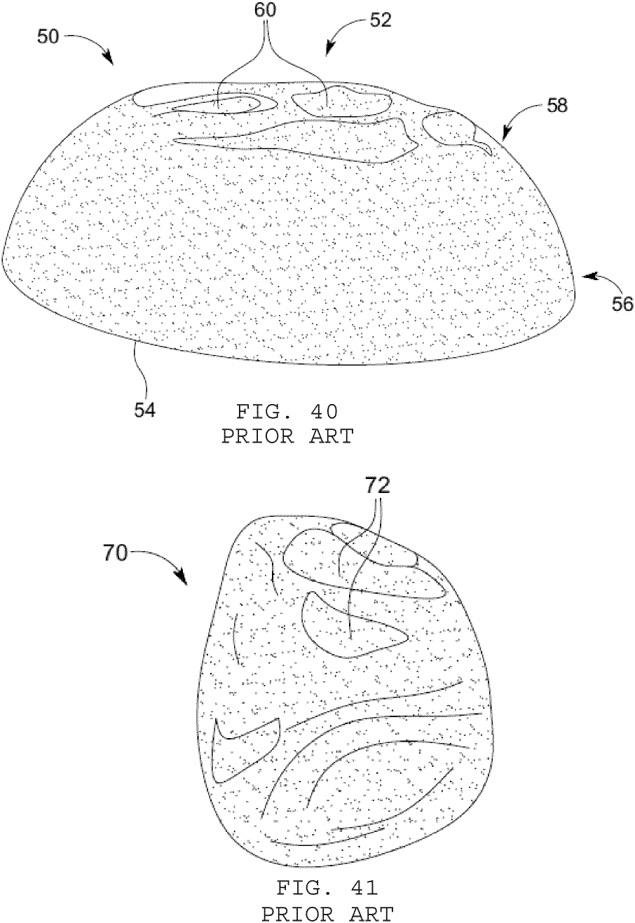

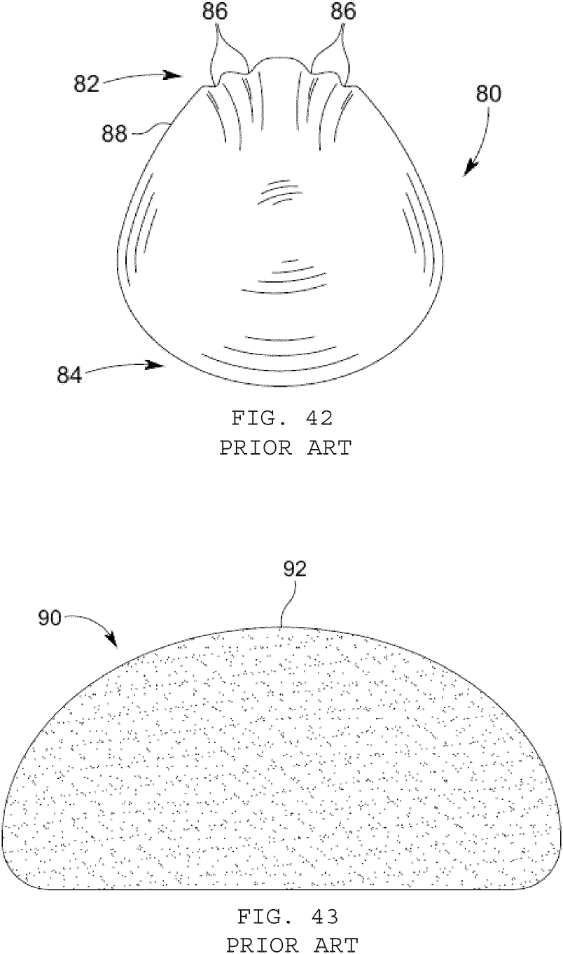

In FIG. 353, the shell 1202 of the implantable prosthetic device 1200 is in a horizontal orientation with the rear wall 1206 of the shell 1202 overlying a horizontal surface. FIG. 36 shows the implantable prosthetic device 1200 of FIG. 35 after the shell 1202 has been rotated 90 degrees in a clockwise direction relative to the horizontal orientation shown in FIG. 35. In FIG. 36, the shell 1202 may be described as being in a vertical orientation. The scaffold 1216 that is disposed inside the shell 1202 of the implantable device 1200 preferably provides an internal reinforcement for the shell that maintains the shape and form stability of the shell. The rear wall 1206 of the shell, reinforced by the scaffold 1216, extends along a vertical axis and the shell does not lose its shape as occurs with the prior art implants shown in FIGS. 40-43 of the present patent application.

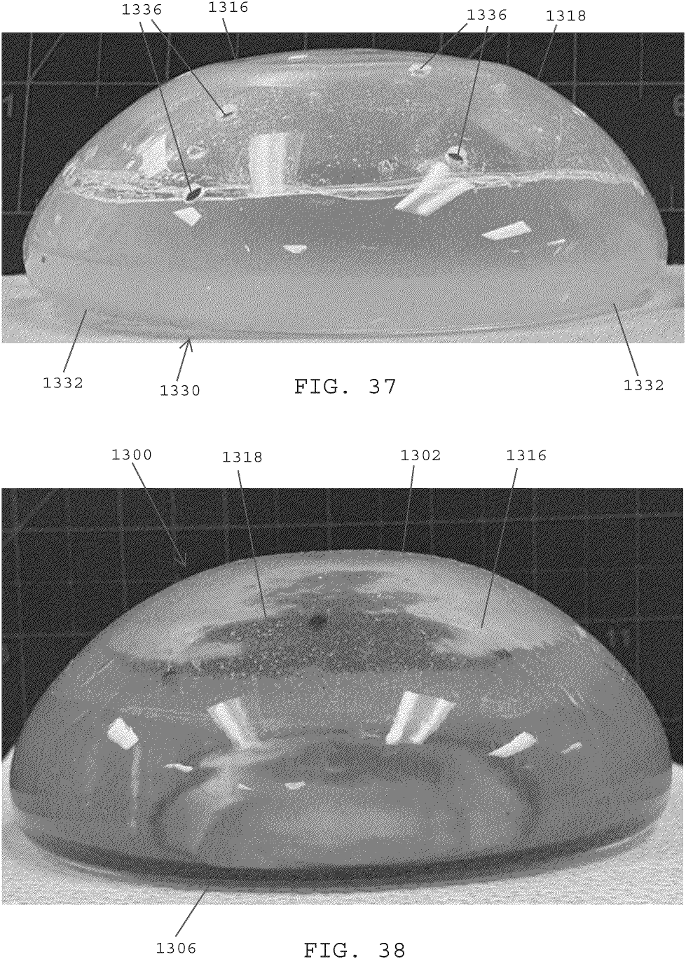

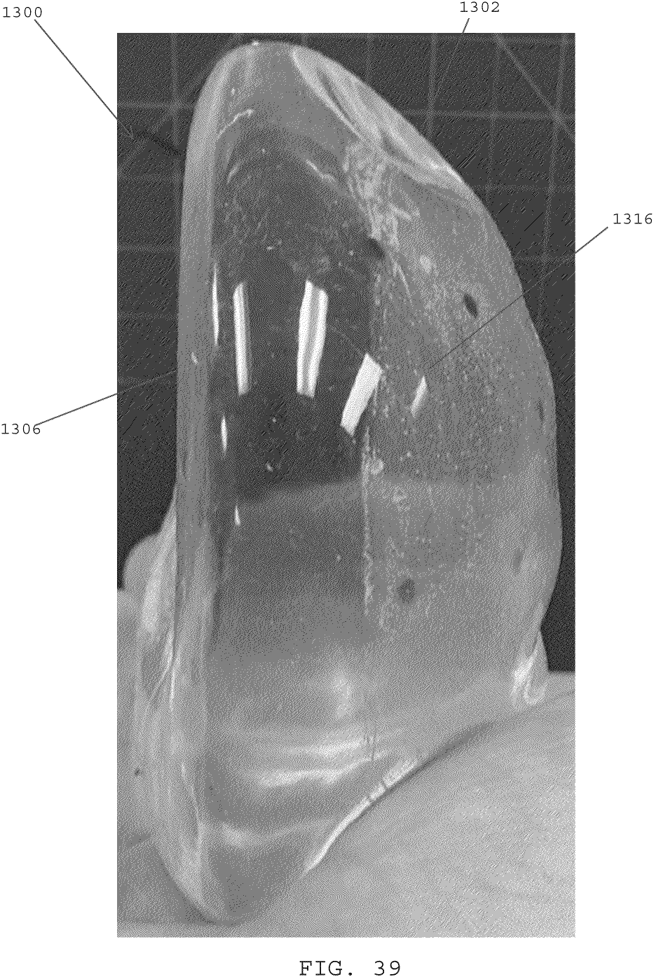

Referring to FIG. 37, in one embodiment, a scaffold 1316 for an implantable prosthetic device preferably includes a wall 1318 having a dome shape. The wall 1318 may mirror the shape of a hollow hemisphere. The scaffold 1316 preferably includes an open base 1330 that is surrounded by a circular, free edge 1332 of the dome-shaped wall 1318 of the scaffold. In one embodiment, a plurality of openings 1336 are formed in the curved wall 1318. The openings 1336 in the wall 1318 preferably extend between an outer surface and an inner surface of the curved wall 1318.

Referring to FIG. 38, in one embodiment, the scaffold 1316 is disposed within an outer shell 1302 of an implantable prosthetic device 1300. The circular, free edge 1332 of the curved wall 1318 of the scaffold 1316 that surrounds the open base 1330 (FIG. 37) is preferably juxtaposed with the rear wall 1306 of the shell 1302. In one embodiment, the shell 1302 of the implantable device 1300 is preferably filled with a biocompatible filler material such as a silicone gel. The biocompatible filler material desirably completely fills the interior chamber of the shell 1302 and is preferably adherent to one or more of the inner and outer surfaces of the wall 1318 of the scaffold 1316 that is disposed within the shell 1302.

In FIG. 38, the shell 1302 of the implantable prosthetic device 1300 is in a horizontal orientation with the rear wall 1306 of the shell 1302 overlying a horizontal surface. FIG. 39 shows the implantable prosthetic device 1300 of FIG. 38 after the shell 1302 has been rotated 90 degrees in a clockwise direction relative to the horizontal orientation shown in FIG. 38. In FIG. 39, the shell 1302 may be described as being in a vertical orientation. The scaffold 1316 that is disposed inside the shell 1302 of the implantable device 1300 preferably provides an internal reinforcement for the shell that maintains the shape and form stability of the shell. The rear wall 1306 of the shell, reinforced by the scaffold 1316, extends along a vertical axis and the shell does not lose its shape as occurs with the prior art implants shown in FIGS. 40-43 of the present patent application.

While the foregoing is directed to embodiments of the present invention, other and further embodiments of the invention may be devised without departing from the basic scope thereof, which is only limited by the scope of the claims that follow. For example, the present invention contemplates that any of the features shown in any of the embodiments described herein, or incorporated by reference herein, may be incorporated with any of the features shown in any of the other embodiments described herein, or incorporated by reference herein, and still fall within the scope of the present invention.

What is claimed is:

1. An implantable prosthetic device comprising:
a shell made of a biocompatible elastomeric material, said shell having a front portion and a base that surround an interior volume of said shell;
a scaffold disposed within the interior volume of said shell, said scaffold having an inner surface that faces toward said base of said shell and an outer surface that faces toward said front portion of said shell;
a biocompatible filler material disposed within the interior volume of said shell that surrounds the inner and outer surfaces of said scaffold.

2. The implantable prosthetic device as claimed in claim 1, wherein said biocompatible elastomeric material of said shell comprises silicone.

3. The implantable prosthetic device as claimed in claim 1, wherein said biocompatible filler material disposed within the interior volume of said shell comprises a cohesive filler material or a silicone gel.

4. The implantable prosthetic device as claimed in claim 1, wherein said biocompatible filler material disposed within the interior volume of said shell is adhered to at least a portion of the inner surface or the outer surface of said scaffold.

5. The implantable prosthetic device as claimed in claim 1, wherein the inner surface of said scaffold is concave and the outer surface of said scaffold is convexly curved.

6. The implantable prosthetic device as claimed in claim 5, wherein the convexly curved outer surface of said scaffold mirrors the shape of said front portion of said shell.

7. The implantable prosthetic device as claimed in claim 1, wherein said scaffold comprises a wall having one or more openings formed therein that extend from the inner surface of said scaffold wall to the outer surface of said scaffold wall.

8. The implantable prosthetic device as claimed in claim 1, wherein said scaffold is attached to said base of said shell.

9. The implantable prosthetic device as claimed in claim 1, wherein said scaffold has the general shape of a hemisphere having an open base.

10. The implantable prosthetic device as claimed in claim 9, wherein said scaffold has a lower end including a circular, free edge that surrounds an opening in said open base of said scaffold.

11. The implantable prosthetic device as claimed in claim 10, wherein said circular, free edge of said scaffold is juxtaposed with said base of said shell.

12. The implantable prosthetic device as claimed in claim 11, wherein said circular, free edge of said scaffold is attached to said base of said shell.

13. The implantable prosthetic device as claimed in claim 1, wherein said front portion of said shell includes an apex and a dome that extends between said apex and said base of said shell, and wherein said scaffold has a shape that mirrors the shape of said dome of said shell.

14. The implantable prosthetic device as claimed in claim 1, further comprising a second scaffold that is nested within said first scaffold, wherein said second scaffold is located between the inner surface of said first scaffold and said base of said shell.

15. The implantable prosthetic device as claimed in claim 14, wherein said second scaffold has a curved wall with one or more openings formed therein that extend from an inner surface to an outer surface of said curved wall of said second scaffold.

16. The implantable prosthetic device as claimed in claim 14, wherein said biocompatible filler material is in contact with at least one of said inner and outer surfaces of said curved wall of said second scaffold.

17. The implantable prosthetic device as claimed in claim 14, wherein said front portion of said shell has a shell wall thickness, said first scaffold has a first scaffold wall thickness, and said second scaffold has a second scaffold wall thickness, wherein said second scaffold wall thickness is greater than said first scaffold wall thickness, and wherein said first scaffold wall thickness is greater than said shell wall thickness.

18. The implantable prosthetic device as claimed in claim 14, wherein said second scaffold has a geometric shape that is different than the geometric shape of said first scaffold or the geometric shape of said front portion of said shell.

19. The implantable prosthetic device as claimed in claim 14, wherein said first scaffold has a geometric shape that is different than the geometric shape of said second scaffold or the geometric shape of said front portion of said shell.

20. The implantable prosthetic device as claimed in claim 1, wherein said shell has a shell wall thickness and said scaffold has a scaffold wall thickness that is greater than the shell wall thickness.

21. The implantable prosthetic device as claimed in claim 1, wherein said scaffold has an apex, a radius, and a dome that extends between said apex of said scaffold and said radius of said scaffold, and wherein said scaffold has a wall that is thicker at said apex of said scaffold and thinner at said radius of said scaffold.

22. The implantable prosthetic device as claimed in claim 1, wherein said shell and said scaffold comprise silicone.

23. The implantable prosthetic device as claimed in claim 1, wherein said implantable prosthetic device is a breast implant.

24. An implantable prosthetic device comprising:
a silicone shell having a front wall portion and a base that surround an interior volume of said silicone shell;
a silicone scaffold disposed within the interior volume of said silicone shell, said silicone scaffold having a concave inner surface that faces toward said base of said silicone shell and a convexly curved outer surface that faces toward said front wall portion of said silicone shell;
a silicone gel disposed within the interior volume of said silicone shell that fills said silicone shell and surrounds said silicone scaffold.

25. The implantable prosthetic device as claimed in claim 24, wherein said silicone scaffold has one or more openings formed therein that extend from the concave inner surface to the convexly curved outer surface of said silicone scaffold.

26. The implantable prosthetic device as claimed in claim 24, wherein said silicone scaffold is a hemisphere having an open base and a lower, free edge that surrounds said open base.

27. The implantable prosthetic device as claimed in claim 26, wherein said lower, free edge of said silicone scaffold is juxtaposed with said base of said silicone shell.

28. The implantable prosthetic device as claimed in claim 26, wherein said lower, free edge of said silicone scaffold is attached to said base of said silicone shell.

29. The implantable prosthetic device as claimed in claim 24, further comprising a second silicone scaffold that is nested within said first silicone scaffold and that is located between said concave inner surface of said first silicone scaffold and said base of said silicone shell.

30. The implantable prosthetic device as claimed in claim 24, wherein said front wall of said silicone shell comprises an apex and a dome that extends between said apex and said base of said silicone shell, and wherein said silicone scaffold has a geometric shape that mirrors the geometric shape of said dome of said silicone shell.

31. The implantable prosthetic device as claimed in claim 24, wherein said front wall portion of said silicone shell has a first wall thickness and said silicone scaffold has a second wall thickness that is greater than the first wall thickness.
